(19) United States
(12) Patent Application Publication
(10) Pub. No.: US 2013/0060022 A1
(43) Pub. Date: Mar. 7, 2013

(54) Compounds useful for treating neurodegenerative disorders

(71) Applicants: Brian Scott Bronk, East Lyme, CT (US); Wesley Francis Austin, Cambridge, MA (US); Steffen Phillip Creaser, Cambridge, MA (US); Nathan Oliver Fuller, Somerville, MA (US); Jed Lee Hubbs, Cambridge, MA (US); Jeffrey Lee Ives, Chester, CT (US); Ruichao Shen, West Roxbury, MA (US)

(72) Inventors: Brian Scott Bronk, East Lyme, CT (US); Wesley Francis Austin, Cambridge, MA (US); Steffen Phillip Creaser, Cambridge, MA (US); Nathan Oliver Fuller, Somerville, MA (US); Jed Lee Hubbs, Cambridge, MA (US); Jeffrey Lee Ives, Chester, CT (US); Ruichao Shen, West Roxbury, MA (US)

(73) Assignee: Satori Pharmaceuticals, Inc., Cambridge, MA (US)

(21) Appl. No.: 13/605,970

(22) Filed: Sep. 6, 2012

Related U.S. Application Data
(60) Provisional application No. 61/532,059, filed on Sep. 7, 2011.

(51) Int. Cl. C07J 71/00

(52) U.S. Cl. 540/48

(57) ABSTRACT

The present invention provides compounds of formula I:

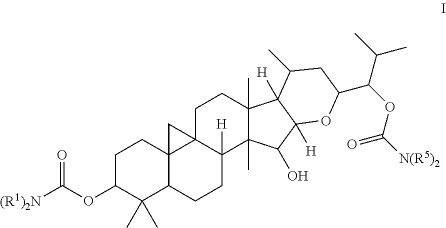

or a pharmaceutically acceptable salt thereof, wherein R¹ and R⁵ are as defined and described herein, compositions thereof, and methods of using the same.

### CROSS REFERENCE TO RELATED APPLICATION

This application claims the benefit of U.S. Provisional Application Ser. No. 61/532,059, filed Sep. 7, 2011, the disclosure of which is incorporated in its entirety herein by reference.

### TECHNICAL FIELD OF INVENTION

The present invention relates to pharmaceutically active compounds useful for treating, or lessening the severity of, neurodegenerative disorders.

### BACKGROUND OF THE INVENTION

The central role of the long form of amyloid beta-peptide, in particular Aβ(1-42), in Alzheimer's disease has been established through a variety of histopathological, genetic and biochemical studies. See Selkoe, D J, Physiol. Rev. 2001, 81:741-766, Alzheimer's disease: genes, proteins, and therapy, and Younkin S G, J. Physiol. Paris. 1998, 92:289-92, The role of A beta 42 in Alzheimer's disease. Specifically, it has been found that deposition in the brain of Aβ(1-42) is an early and invariant feature of all forms of Alzheimer's disease. In fact, this occurs before a diagnosis of Alzheimer's disease is possible and before the deposition of the shorter primary form of A-beta, Aβ(1-40). See Parvathy S, et al., Arch. Neurol. 2001, 58:2025-32, Correlation between Abetax-40-, Abetax-42-, and Abetax-43-containing amyloid plaques and cognitive decline. Further implication of Aβ(1-42) in disease etiology comes from the observation that mutations in presenilin (gamma secretase) genes associated with early onset familial forms of Alzheimer's disease uniformly result in increased levels of Aβ(1-42). See Ishii K., et al., Neurosci. Lett. 1997, 228:17-20, Increased A beta 42(43)-plaque deposition in early-onset familial Alzheimer's disease brains with the deletion of exon 9 and the missense point mutation (H163R) in the PS-1 gene. Additional mutations in the amyloid precursor protein APP raise total Aβ and in some cases raise Aβ(1-42) alone. See Kosaka T, et al., Neurology, 48:741-5, The beta APP717 Alzheimer mutation increases the percentage of plasma amyloid-beta protein ending at A beta42(43). Although the various APP mutations may influence the type, quantity, and location of Aβ deposited, it has been found that the predominant and initial species deposited in the brain parenchyma is long Aβ (Mann). See Mann D M, et al., Am. J. Pathol. 1996, 148:1257-66, “Predominant deposition of amyloid-beta 42(43) in plaques in cases of Alzheimer's disease and hereditary cerebral hemorrhage associated with mutations in the amyloid precursor protein gene”.

In early deposits of Aβ, when most deposited protein is in the form of amorphous or diffuse plaques, virtually all of the Aβ is of the long form. See Gravina S A, et al., J. Biol. Chem., 270:7013-6, Amyloid beta protein (A beta) in Alzheimer's disease brain. Biochemical and immunocytochemical analysis with antibodies specific for forms ending at A beta 40 or A beta 42(43); Iwatsubo T, et al., Am. J. Pathol. 1996, 149:1823-30, Full-length amyloid-beta (1-42(43)) and amino-terminally modified and truncated amyloid-beta 42(43) deposit in diffuse plaques; and Roher A E, et al., Proc. Natl. Acad. Sci. USA. 1993, 90:10836-40, beta-Amyloid-(1-42) is a major component of cerebrovascular amyloid deposits: implications for the pathology of Alzheimer disease. These initial deposits of Aβ(1-42) then are able to seed the further deposition of both long and short forms of Aβ. See Tamaoka A, et al., Biochem. Biophys. Res. Commun. 1994, 205:834-42, Biochemical evidence for the long-tail form (A beta 1-42/43) of amyloid beta protein as a seed molecule in cerebral deposits of Alzheimer's disease.

In transgenic animals expressing Aβ, deposits were associated with elevated levels of Aβ(1-42), and the pattern of deposition is similar to that seen in human disease with Aβ(1-42) being deposited early followed by deposition of Aβ(1-40). See Rockenstein E, et al., J. Neurosci. Res. 2001, 66:573-82, Early formation of mature amyloid-beta protein deposits in a mutant APP transgenic model depends on levels of Abeta(1-42); and Terai K, et al., Neuroscience 2001, 104:299-310, beta-Amyloid deposits in transgenic mice expressing human beta-amyloid precursor protein have the same characteristics as those in Alzheimer's disease. Similar patterns and timing of deposition are seen in Down's syndrome patients in which Aβ expression is elevated and deposition is accelerated. See Iwatsubo T., et al., Ann. Neurol. 1995, 37:294-9, Amyloid beta protein (A beta) deposition: A beta 42(43) precedes A beta 40 in Down syndrome.

Accordingly, selective lowering of Aβ(1-42) thus emerges as a disease-specific strategy for reducing the amyloid forming potential of all forms of Aβ, slowing or stopping the formation of new deposits of Aβ, inhibiting the formation of soluble toxic oligomers of Aβ, and thereby slowing or halting the progression of neurodegeneration.

### SUMMARY OF THE INVENTION

As described herein, the present invention provides compounds useful for treating or lessening the severity of a neurodegenerative disorder. The present invention also provides methods of treating or lessening the severity of such disorders wherein said method comprises administering to a patient a compound of the present invention, or composition thereof. Said method is useful for treating or lessening the severity of, for example, Alzheimer's disease.

### DETAILED DESCRIPTION OF CERTAIN EMBODIMENTS OF THE INVENTION

### 1. General Description of Compounds of the Invention

According to one embodiment, the present invention provides a compound of formula I:

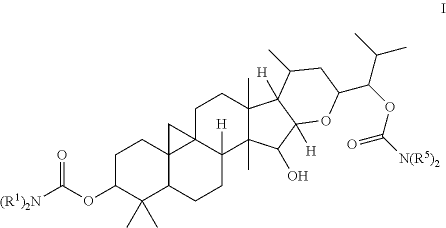

or a pharmaceutically acceptable salt thereof, wherein:
each R¹ is independently hydrogen, straight or branched C₁₋₆ alkyl, 3-6 membered cycloalkyl, or 3-6 membered saturated heterocyclyl having 1-2 heteroatoms independently selected from oxygen, nitrogen, or sulfur, wherein each R¹ is optionally substituted with 1-4 R² groups, or:
   two R¹ groups are taken together to form a 3-7 membered heterocyclic ring having 0-2 heteroatoms independently selected from oxygen, nitrogen, or sulfur in addition to the nitrogen atom where R¹ groups are attached, wherein the ring is optionally substituted with 1-3 R² groups; or
   two R¹ groups are taken together to form a 6-10 membered fused or spiro heterocyclic ring having 0-4 heteroatoms independently selected from oxygen, nitrogen, or sulfur in addition to the nitrogen atom where R¹ groups are attached, wherein the ring is optionally substituted with 1-3 R² groups;
each R² is independently R, halogen, —C(O)N(R)₂, —N(R)₂, —OR, —NR³S(O)₂R³, oxo, —C(O)OR, C₁₋₃ alkyl optionally substituted with 1-3 R³ groups, 3-6 membered cycloalkyl, 3-6 membered saturated heterocyclyl having 1-2 heteroatoms independently selected from oxygen, nitrogen, or sulfur, wherein the cycloalkyl and heterocyclyl are each optionally substituted with 1-3 R³, or:
   two R² groups on the same carbon atom are taken together to form an optionally substituted 3-6 membered saturated carbocyclic or a 3-7 membered heterocyclic ring having 1-2 heteroatoms independently selected from oxygen, nitrogen, or sulfur;
each R³ is independently hydrogen, halogen, —OR, 3-6 membered cycloalkyl, —N(R)₂, C₁₋₃ alkyl optionally substituted with 1-3 R⁴ groups, or:
   two R³ groups on the same carbon atom are taken together to form an optionally substituted 3-6 membered saturated carbocyclic or a 3-7 membered heterocyclic ring having 1-2 heteroatoms independently selected from oxygen, nitrogen, or sulfur;
each R⁴ is independently hydrogen, halogen, C₁₋₃ alkyl optionally substituted with 1-3 —OR groups, 3-6 membered cycloalkyl, or:
   two R⁴ groups on the same carbon atom are taken together to form an optionally substituted 3-6 membered saturated carbocyclic or a 3-7 membered heterocyclic ring having 1-2 heteroatoms independently selected from oxygen, nitrogen, or sulfur;
each R⁵ is independently hydrogen or C₁₋₃ alkyl, or:
   two R⁵ groups on the same nitrogen atom are taken together to form an optionally substituted 3-5 membered heterocyclic ring; and
each R is independently hydrogen, C₁₋₄ aliphatic, or:
   two R groups on the same nitrogen atom are taken together to form an optionally substituted 4-8 membered saturated or partially unsaturated ring.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-20 aliphatic carbon atoms. In some embodiments, aliphatic groups contain 1-6 aliphatic carbon atoms. In yet other embodiments aliphatic groups contain 1-4 aliphatic carbon atoms. In some embodiments, “cycloaliphatic” (or “carbocycle” or “cycloalkyl”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl. In other embodiments, an aliphatic group may have two geminal hydrogen atoms replaced with oxo (a bivalent carbonyl oxygen atom ═O), or a ring-forming substituent, such as —O-(straight or branched alkylene or alkylene)-O— to form an acetal or ketal. The term “alkylene,” as used herein, refers to a bivalent straight or branched saturated or unsaturated hydrocarbon chain. In some embodiments, an alkylene group is saturated.

In certain embodiments, exemplary aliphatic groups include, but are not limited to, ethynyl, 2-propynyl, 1-propenyl, 2-butenyl, 1,3-butadienyl, 2-pentenyl, vinyl (ethenyl), allyl, isopropenyl, methyl, ethyl, propyl, isopropyl, butyl, isobutyl, sec-butyl, tert-butyl, pentyl, isopentyl, sec-pentyl, neo-pentyl, tert-pentyl, cyclopentyl, hexyl, isohexyl, sec-hexyl, cyclohexyl, 2-methylpentyl, tert-hexyl, 2,3-dimethylbutyl, 3,3-dimethylbutyl, 1,3-dimethylbutyl, and 2,3-dimethyl but-2-yl.

The term “heterocycle,” “heterocyclyl,” “heterocycloaliphatic,” or “heterocyclic” as used herein means non-aromatic, monocyclic, bicyclic, or tricyclic ring systems in which one or more ring members is an independently selected heteroatom. In some embodiments, the “heterocycle,” “heterocyclyl,” “heterocycloaliphatic,” or “heterocyclic” group has three to fourteen ring members in which one or more ring members is a heteroatom independently selected from oxygen, sulfur, nitrogen, or phosphorus, and each ring in the system contains 3 to 7 ring members.

A heterocyclic ring can be attached to its pendant group at any heteroatom or carbon atom that results in a stable structure and, when specified, any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated,” as used herein, means that a moiety has one or more units of unsaturation.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “aryl” used alone or as part of a larger moiety as in “aralkyl,” “aralkoxy,” or “aryloxyalkyl,” refers to monocyclic, bicyclic, and tricyclic ring systems having a total of five to fourteen ring members, wherein one or more ring in the system is aromatic and wherein each ring in the system contains 3 to 7 ring members. The term “aryl” may be used interchangeably with the term “aryl ring”. The term “aryl” also refers to heteroaryl ring systems as defined hereinbelow. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The term “heteroaryl,” used alone or as part of a larger moiety as in “heteroaralkyl” or “heteroarylalkoxy,” refers to monocyclic, bicyclic, and tricyclic ring systems having a total of five to fourteen ring members, wherein one or more ring in the system is aromatic, one or more ring in the system contains one or more heteroatoms, and wherein each ring in the system contains 3 to 7 ring members. The term “heteroaryl” may be used interchangeably with the term “heteroaryl ring” or the term “heteroaromatic”. Heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl.

The terms “heteroaryl” and “heteroar-,” as used herein, also include groups in which a heteroaromatic ring is fused to one or more aryl, cycloaliphatic, or heterocyclyl rings. Examplary heteroaryl rings include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄—CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched)alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, —(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, and ═C(R*)₂, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this invention. Such compounds are useful, for example, as analytical tools or probes in biological assays.

### 3. Description of Exemplary Compounds

As described generally above, the present invention provides a compound of formula I:

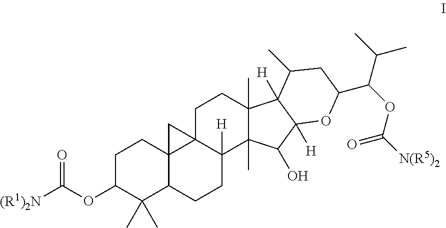

or a pharmaceutically acceptable salt thereof, wherein each variable is defined above and described in classes and subclasses above and herein.

In certain embodiments, the present invention provides a compound of formula I having the stereochemistry depicted in formula I-a, below:

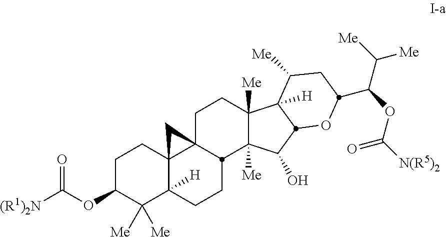

or a pharmaceutically acceptable salt thereof, wherein each variable is defined above and described in classes and subclasses above and herein for compounds of formula I.

As defined generally above, each R¹ is independently hydrogen, straight or branched C₁₋₆ alkyl, 3-6 membered cycloalkyl, or 3-6 membered saturated heterocyclyl having 1-2 heteroatoms independently selected from oxygen, nitrogen, or sulfur, wherein each R¹ is optionally substituted with 1-4 R² groups, or:
two R¹ groups are taken together to form a 3-7 membered heterocyclic ring having 0-2 heteroatoms independently selected from oxygen, nitrogen, or sulfur in addition to the nitrogen atom where R¹ groups are attached, wherein the ring is optionally substituted with 1-3 R² groups;
   wherein each R² is as defined above and described herein.

In certain embodiments, each R¹ is hydrogen.

In certain embodiments, each R¹ is independently straight or branched C₁₋₆ alkyl, 3-6 membered cycloalkyl, or 3-6 membered saturated heterocyclyl having 1-2 heteroatoms independently selected from oxygen, nitrogen, or sulfur, wherein each R¹ is optionally substituted with 1-4 R² groups, or:
two R¹ groups are taken together to form a 3-7 membered heterocyclic ring having 0-2 heteroatoms independently selected from oxygen, nitrogen, or sulfur in addition to the nitrogen atom where R¹ groups are attached, wherein the ring is optionally substituted with 1-3 R² groups; or
two R¹ groups are taken together to form a 6-10 membered fused or spiro heterocyclic ring having 0-4 heteroatoms independently selected from oxygen, nitrogen, or sulfur in addition to the nitrogen atom where R¹ groups are attached, wherein the ring is optionally substituted with 1-3 R² groups;
   wherein each R² is as defined above and described herein.

In certain embodiments, each R¹ is independently straight or branched C₁₋₆ alkyl optionally substituted with 1-4 R² groups.

In certain embodiments, each R¹ is independently straight or branched C₁₋₆ alkyl. In certain embodiments, each R¹ is independently straight or branched C₁₋₅ alkyl. In certain embodiments, each R¹ is independently straight or branched C₁₋₄ alkyl. In certain embodiments, each R¹ is independently straight or branched C₁₋₃ alkyl. In certain embodiments, each R¹ is ethyl. In certain embodiments, each R¹ is methyl.

In certain embodiments, each R¹ is independently straight or branched C₁₋₆ alkyl substituted with 1-4 R² groups wherein each R² is as defined above and described herein. In certain embodiments, each R¹ is independently straight or branched C₁₋₅ alkyl substituted with 1-4 R² groups wherein each R² is as defined above and described herein. In certain embodiments, each R¹ is independently straight or branched C₁₋₄ alkyl substituted with 1-4 R² groups wherein each R² is as defined above and described herein. In certain embodiments, each R¹ is independently straight or branched C₁₋₃ alkyl substituted with 1-4 R² groups wherein each R² is as defined above and described herein. In certain embodiments, each R¹ is ethyl substituted with 1-4 R² groups wherein each R² is as defined above and described herein. In certain embodiments, each R¹ is methyl substituted with 1-3 R² groups wherein each R² is as defined above and described herein.

In certain embodiments, each R¹ is independently 3-6 membered cycloalkyl optionally substituted with 1-4 R² groups.

In certain embodiments, each R¹ is independently 3-6 membered cycloalkyl. In certain embodiments, each R¹ is independently 3-5 membered cycloalkyl. In certain embodiments, each R¹ is independently 3-4 membered cycloalkyl.

In certain embodiments, each R¹ is cyclohexyl. In certain embodiments, each R¹ is cyclopentyl. In certain embodiments, each R¹ is cyclobutyl. In certain embodiments, each R¹ is cyclopropyl.

In certain embodiments, each R¹ is independently 3-6 membered cycloalkyl substituted with 1-4 R² groups wherein each R² is as defined above and described herein. In certain embodiments, each R¹ is independently 3-5 membered cycloalkyl substituted with 1-4 R² groups wherein each R² is as defined above and described herein. In certain embodiments, each R¹ is independently 3-4 membered cycloalkyl substituted with 1-4 R² groups wherein each R² is as defined above and described herein.

In certain embodiments, each R¹ is cyclohexyl substituted with 1-4 R² groups wherein each R² is as defined above and described herein. In certain embodiments, each R¹ is cyclopentyl substituted with 1-4 R² groups wherein each R² is as defined above and described herein. In certain embodiments, each R¹ is cyclobutyl substituted with 1-4 R² groups wherein each R² is as defined above and described herein. In certain embodiments, each R¹ is cyclopropyl substituted with 1-4 R² groups wherein each R² is as defined above and described herein.

In certain embodiments, each R¹ is independently 3-6 membered saturated heterocyclyl having 1-2 heteroatoms independently selected from oxygen, nitrogen, or sulfur wherein each R¹ is optionally substituted with 1-4 R² groups wherein each R² is as defined above and described herein.

In certain embodiments, each R¹ is independently 3-6 membered saturated heterocyclyl having 1-2 heteroatoms independently selected from oxygen, nitrogen, or sulfur. In certain embodiments, each R¹ is independently 3-5 membered saturated heterocyclyl having 1-2 heteroatoms independently selected from oxygen, nitrogen, or sulfur. In certain embodiments, each R¹ is independently 3-4 membered saturated heterocyclyl having 1-2 heteroatoms independently selected from oxygen, nitrogen, or sulfur. In certain embodiments, each R¹ is independently 6 membered saturated heterocyclyl having 1-2 heteroatoms independently selected from oxygen, nitrogen, or sulfur. In certain embodiments, each R¹ is independently 5 membered saturated heterocyclyl having 1-2 heteroatoms independently selected from oxygen, nitrogen, or sulfur. In certain embodiments, each R¹ is independently 4 membered saturated heterocyclyl having 1-2 heteroatoms independently selected from oxygen, nitrogen, or sulfur. In certain embodiments, each R¹ is independently 3 membered saturated heterocyclyl having 1-2 heteroatoms independently selected from oxygen, nitrogen, or sulfur.

In certain embodiments, each R¹ is independently piperidinyl, pyrrolidinyl, azetidinyl, or aziridinyl. In certain embodiments, each R¹ is independently tetrahydro-2H-pyranyl, tetrahydrofuranyl, or oxetanyl.

In certain embodiments, each R¹ is independently 3-6 membered saturated heterocyclyl having 1-2 heteroatoms independently selected from oxygen, nitrogen, or sulfur wherein each R¹ is substituted with 1-4 R² groups wherein each R² is as defined above and described herein. In certain embodiments, each R¹ is independently 3-5 membered saturated heterocyclyl having 1-2 heteroatoms independently selected from oxygen, nitrogen, or sulfur wherein each R¹ is substituted with 1-4 R² groups wherein each R² is as defined above and described herein. In certain embodiments, each R¹ is independently 3-4 membered saturated heterocyclyl having 1-2 heteroatoms independently selected from oxygen, nitrogen, or sulfur wherein each R¹ is substituted with 1-4 R² groups wherein each R² is as defined above and described herein. In certain embodiments, each R¹ is independently 6 membered saturated heterocyclyl having 1-2 heteroatoms independently selected from oxygen, nitrogen, or sulfur wherein each R¹ is substituted with 1-4 R² groups wherein each R² is as defined above and described herein. In certain embodiments, each R¹ is independently 5 membered saturated heterocyclyl having 1-2 heteroatoms independently selected from oxygen, nitrogen, or sulfur wherein each R¹ is substituted with 1-4 R² groups wherein each R² is as defined above and described herein. In certain embodiments, each R¹ is independently 4 membered saturated heterocyclyl having 1-2 heteroatoms independently selected from oxygen, nitrogen, or sulfur wherein each R¹ is substituted with 1-4 R² groups wherein each R² is as defined above and described herein. In certain embodiments, each R¹ is independently 3 membered saturated heterocyclyl having 1-2 heteroatoms independently selected from oxygen, nitrogen, or sulfur wherein each R¹ is substituted with 1-4 R² groups wherein each R² is as defined above and described herein.

In certain embodiments, each R¹ is independently piperidinyl, pyrrolidinyl, azetidinyl, or aziridinyl substituted with 1-4 R² groups wherein each R² is as defined above and described herein. In certain embodiments, each R¹ is independently tetrahydro-2H-pyranyl, tetrahydrofuranyl, or oxetanyl substituted with 1-4 R² groups wherein each R² is as defined above and described herein.

In certain embodiments, two R¹ groups are taken together to form a 3-7 membered heterocyclic ring having 0-2 heteroatoms independently selected from oxygen, nitrogen, or sulfur in addition to the nitrogen atom where R¹ groups are attached, wherein the ring is optionally substituted with 1-3 R² groups and wherein each R² is as defined above and described herein. In certain embodiments, two R¹ groups are taken together to form a 3-6 membered heterocyclic ring having 0-2 heteroatoms independently selected from oxygen, nitrogen, or sulfur in addition to the nitrogen atom where R¹ groups are attached, wherein the ring is optionally substituted with 1-3 R² groups and wherein each R² is as defined above and described herein. In certain embodiments, two R¹ groups are taken together to form a 3-5 membered heterocyclic ring having 0-2 heteroatoms independently selected from oxygen, nitrogen, or sulfur in addition to the nitrogen atom where R¹ groups are attached, wherein the ring is optionally substituted with 1-3 R² groups and wherein each R² is as defined above and described herein. In certain embodiments, two R¹ groups are taken together to form a 3-4 membered heterocyclic ring having 0-2 heteroatoms independently selected from oxygen, nitrogen, or sulfur in addition to the nitrogen atom where R¹ groups are attached, wherein the ring is optionally substituted with 1-3 R² groups and wherein each R² is as defined above and described herein.

In certain embodiments, two R¹ groups are taken together to form a azetidinyl ring optionally substituted with 1-3 R² groups wherein each R² is as defined above and described herein. In certain embodiments, two R¹ groups are taken together to form a pyrrolidinyl ring optionally substituted with 1-3 R² groups wherein each R² is as defined above and described herein. In certain embodiments, two R¹ groups are taken together to form a piperidinyl ring optionally substituted with 1-3 R² groups wherein each R² is as defined above and described herein. In certain embodiments, two R¹ groups are taken together to form a piperazinyl ring optionally substituted with 1-3 R² groups wherein each R² is as defined above and described herein.

In certain embodiments, two R¹ groups are taken together to form a 6-10 membered fused or spiro heterocyclic ring having 0-4 heteroatoms independently selected from oxygen, nitrogen, or sulfur in addition to the nitrogen atom where R¹ groups are attached, wherein the ring is optionally substituted with 1-3 R² groups.

Exemplary —N(R¹)₂ groups are depicted below, wherein each R² is as defined above and described herein:

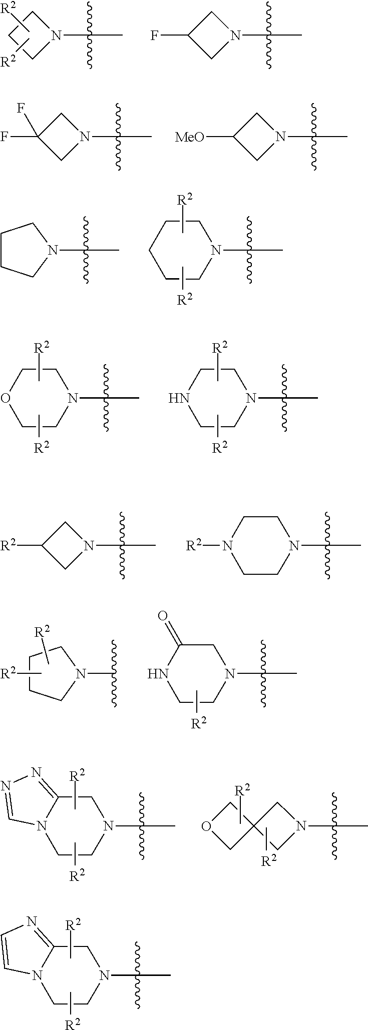

As defined generally above, each R² is independently R, halogen, —C(O)N(R)₂, —N(R)₂, —OR, —NR³S(O)₂R³, oxo, —C(O)OR, C₁₋₃ alkyl optionally substituted with 1-3 R³ groups, 3-6 membered cycloalkyl, 3-6 membered saturated heterocyclyl having 1-2 heteroatoms independently selected from oxygen, nitrogen, or sulfur, wherein the cycloalkyl and heterocyclyl are each optionally substituted with 1-3 R³, or:
two R² groups on the same carbon atom are taken together to form an optionally substituted 3-6 membered saturated carbocyclic or a 3-7 membered heterocyclic ring having 1-2 heteroatoms independently selected from oxygen, nitrogen, or sulfur;
   wherein each of R³ and R is as defined above and described herein.

In certain embodiments, each R² is independently R.

In certain embodiments, each R² is H.

In certain embodiments, each R² is independently halogen. In certain embodiments, each R² is independently —F. In certain embodiments, each R² is independently —Cl. In certain embodiments, each R² is independently —Br. In certain embodiments, each R² is independently —I.

In certain embodiments, each R² is independently —C(O)N(R)₂.

In certain embodiments, each R² is —C(O)NH₂.

In certain embodiments, each R² is independently —N(R)₂.

In certain embodiments, each R² is —NH₂.

In certain embodiments, each R² is independently —OR.

In certain embodiments, each R² is —OH.

In certain embodiments, each R² is independently —NR³S(O)₂R³.

In certain embodiments, each R² is -oxo.

In certain embodiments, each R² is independently —C(O)OR.

In certain embodiments, each R² is —C(O)OH.

In certain embodiments, each R² is independently C₁₋₃ alkyl optionally substituted with 1-3 R³ groups.

In certain embodiments, each R² is independently C₁₋₃ alkyl.

In certain embodiments, each R² is independently C₁₋₃ alkyl substituted with 1-3 R³ groups.

In certain embodiments, each R² is independently 3-6 membered cycloalkyl optionally substituted with 1-3 R³ wherein each R³ is as defined above and described herein. In certain embodiments, each R² is independently cyclohexyl optionally substituted with 1-3 R³ wherein each R³ is as defined above and described herein. In certain embodiments, each R² is independently cyclopentyl optionally substituted with 1-3 R³ wherein each R³ is as defined above and described herein. In certain embodiments, each R² is independently cyclobutyl optionally substituted with 1-3 R³ wherein each R³ is as defined above and described herein. In certain embodiments, each R² is independently cyclopropyl optionally substituted with 1-3 R³ wherein each R³ is as defined above and described herein.

In certain embodiments, each R² is 3-6 membered saturated heterocyclyl having 1-2 heteroatoms independently selected from oxygen, nitrogen, or sulfur, wherein the heterocyclyl are each optionally substituted with 1-3 R³ and wherein each R³ is as defined above and described herein. In certain embodiments, each R² is 3-5 membered saturated heterocyclyl having 1-2 heteroatoms independently selected from oxygen, nitrogen, or sulfur, wherein the heterocyclyl are each optionally substituted with 1-3 R³ and wherein each R³ is as defined above and described herein. In certain embodiments, each R² is 3-4 membered saturated heterocyclyl having 1-2 heteroatoms independently selected from oxygen, nitrogen, or sulfur, wherein the heterocyclyl are each optionally substituted with 1-3 R³ and wherein each R³ is as defined above and described herein.

In certain embodiments, each R² is independently piperidinyl, pyrrolidinyl, azetidinyl, or aziridinyl substituted with 1-3 R³ groups wherein each R³ is as defined above and described herein. In certain embodiments, each R² is independently tetrahydro-2H-pyranyl, tetrahydrofuranyl, or oxetanyl substituted with 1-3 R³ groups wherein each R³ is as defined above and described herein.

In certain embodiments, each R² is independently piperidinyl, pyrrolidinyl, azetidinyl, or aziridinyl. In certain embodiments, each R² is independently tetrahydro-2H-pyranyl, tetrahydrofuranyl, or oxetanyl.

In certain embodiments, two R² groups on the same carbon atom are taken together to form an optionally substituted 3-6 membered saturated carbocyclic or a 3-7 membered heterocyclic ring having 1-2 heteroatoms independently selected from oxygen, nitrogen, or sulfur.

In certain embodiments, two R² groups on the same carbon atom are taken together to form an optionally substituted 3-6 membered saturated carbocyclic ring. In certain embodiments, two R² groups on the same carbon are taken together to form an optionally substituted cyclopropyl, cyclobutyl, cyclopentyl, cyclohexyl, or cycloheptyl ring.

In certain embodiments, two R² groups on the same carbon atom are taken together to form an optionally substituted 3-7 membered heterocyclic ring having 1-2 heteroatoms independently selected from oxygen, nitrogen, or sulfur. In certain embodiments, two R² groups on the same carbon atom are taken together to form an optionally substituted 3-6 membered heterocyclic ring having 1-2 heteroatoms independently selected from oxygen, nitrogen, or sulfur. In certain embodiments, two R² groups on the same carbon atom are taken together to form an optionally substituted 3-5 membered heterocyclic ring having 1-2 heteroatoms independently selected from oxygen, nitrogen, or sulfur. In certain embodiments, two R² groups on the same carbon atom are taken together to form an optionally substituted 3-4 membered heterocyclic ring having 1-2 heteroatoms independently selected from oxygen, nitrogen, or sulfur.

In certain embodiments, two R² groups on the same carbon atom are taken together to form an optionally substituted 3-7 membered heterocyclic ring having one heteroatom independently selected from oxygen, nitrogen, or sulfur. In certain embodiments, two R² groups on the same carbon atom are taken together to form an optionally substituted 3-6 membered heterocyclic ring having one heteroatom independently selected from oxygen, nitrogen, or sulfur. In certain embodiments, two R² groups on the same carbon atom are taken together to form an optionally substituted 3-5 membered heterocyclic ring having one heteroatom independently selected from oxygen, nitrogen, or sulfur. In certain embodiments, two R² groups on the same carbon atom are taken together to form an optionally substituted 3-4 membered heterocyclic ring having one heteroatom independently selected from oxygen, nitrogen, or sulfur.

In certain embodiments, two R² groups on the same carbon are taken together to form an optionally substituted aziridinyl, azetidinyl, pyrrolidinyl, piperidinyl or piperazinyl ring.

In certain embodiments, two R² groups on the same carbon are taken together to form an optionally substituted oxiranyl, oxetanyl, tetrahydrofuranyl or tetrahydro-2H-pyranyl ring.

Exemplary rings formed by two R² on the same carbon atom are depicted below:

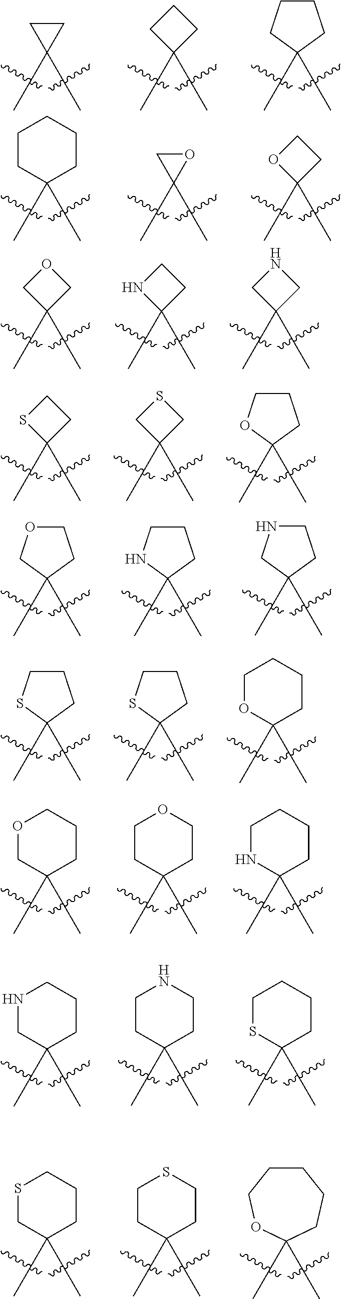

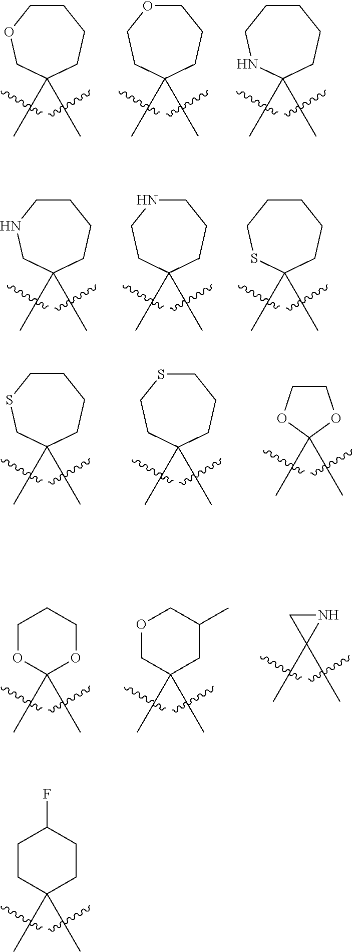

As defined generally above, each R³ is independently hydrogen, halogen, —OR, 3-6 membered cycloalkyl, —N(R)₂, C₁₋₃ alkyl optionally substituted with 1-3 R⁴ groups, or:
two R³ groups on the same carbon atom are taken together to form an optionally substituted 3-6 membered saturated carbocyclic or a 3-7 membered heterocyclic ring having 1-2 heteroatoms independently selected from oxygen, nitrogen, or sulfur;
   wherein each of R⁴ and R is as defined above and described herein.

In certain embodiments, each R³ is hydrogen.

In certain embodiments, each R³ is independently halogen. In certain embodiments, each R³ is independently —F. In certain embodiments, each R³ is independently —Cl. In certain embodiments, each R³ is independently —Br. In certain embodiments, each R³ is independently —I.

In certain embodiments, each R³ is independently —OR.

In certain embodiments, each R³ is —OH.

In certain embodiments, each R³ is independently —N(R)₂.

In certain embodiments, each R³ is —NH₂.

In certain embodiments, each R³ is independently 3-6 membered cycloalkyl.

In certain embodiments, each R³ is independently cyclohexyl, cyclopentyl, cyclobutyl or cyclopropyl.

In certain embodiments, each R³ is independently C₁₋₃ alkyl optionally substituted with 1-3 R⁴ groups.

In certain embodiments, each R³ is independently C₁₋₃ alkyl.

In certain embodiments, each R³ is independently C₁₋₃ alkyl substituted with 1-3 R⁴ groups.

In certain embodiments, two R³ groups on the same carbon atom are taken together to form an optionally substituted 3-6 membered saturated carbocyclic or a 3-7 membered heterocyclic ring having 1-2 heteroatoms independently selected from oxygen, nitrogen, or sulfur.

In certain embodiments, two R³ groups on the same carbon atom are taken together to form an optionally substituted 3-6 membered saturated carbocyclic ring. In certain embodiments, two R³ groups on the same carbon are taken together to form an optionally substituted cyclopropyl, cyclobutyl, cyclopentyl, cyclohexyl, or cycloheptyl ring.

In certain embodiments, two R³ groups on the same carbon atom are taken together to form an optionally substituted 3-7 membered heterocyclic ring having 1-2 heteroatoms independently selected from oxygen, nitrogen, or sulfur. In certain embodiments, two R³ groups on the same carbon atom are taken together to form an optionally substituted 3-6 membered heterocyclic ring having 1-2 heteroatoms independently selected from oxygen, nitrogen, or sulfur. In certain embodiments, two R³ groups on the same carbon atom are taken together to form an optionally substituted 3-5 membered heterocyclic ring having 1-2 heteroatoms independently selected from oxygen, nitrogen, or sulfur. In certain embodiments, two R³ groups on the same carbon atom are taken together to form an optionally substituted 3-4 membered heterocyclic ring having 1-2 heteroatoms independently selected from oxygen, nitrogen, or sulfur.

In certain embodiments, two R³ groups on the same carbon atom are taken together to form an optionally substituted 3-7 membered heterocyclic ring having one heteroatom independently selected from oxygen, nitrogen, or sulfur. In certain embodiments, two R³ groups on the same carbon atom are taken together to form an optionally substituted 3-6 membered heterocyclic ring having one heteroatom independently selected from oxygen, nitrogen, or sulfur. In certain embodiments, two R³ groups on the same carbon atom are taken together to form an optionally substituted 3-5 membered heterocyclic ring having one heteroatom independently selected from oxygen, nitrogen, or sulfur. In certain embodiments, two R³ groups on the same carbon atom are taken together to form an optionally substituted 3-4 membered heterocyclic ring having one heteroatom independently selected from oxygen, nitrogen, or sulfur.

In certain embodiments, two R³ groups on the same carbon are taken together to form an optionally substituted aziridinyl, azetidinyl, pyrrolidinyl, piperidinyl or piperazinyl ring.

In certain embodiments, two R³ groups on the same carbon are taken together to form an optionally substituted oxiranyl, oxetanyl, tetrahydrofuranyl or tetrahydro-2H-pyranyl ring.

Exemplary rings formed by two R³ on the same carbon atom are depicted below:

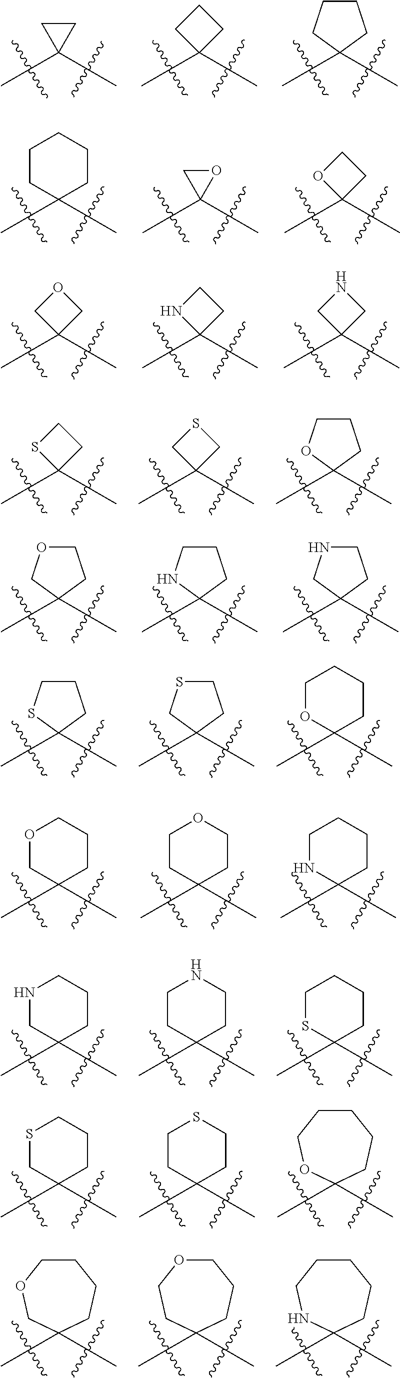

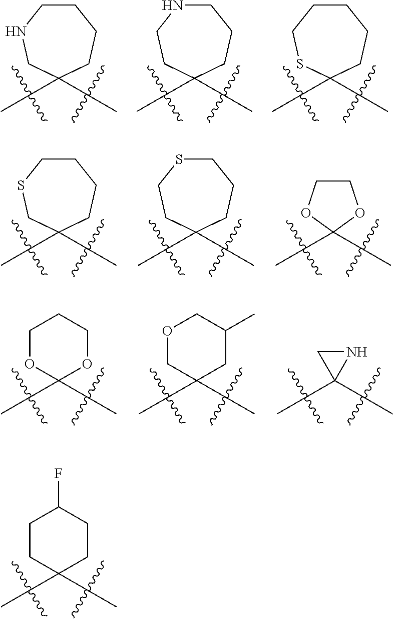

As defined generally above, each R⁴ is independently hydrogen, halogen, C₁₋₃ alkyl optionally substituted with 1-3 —OR groups, 3-6 membered cycloalkyl, or:
two R⁴ groups on the same carbon atom are taken together to form an optionally substituted 3-6 membered saturated carbocyclic or a 3-7 membered heterocyclic ring having 1-2 heteroatoms independently selected from oxygen, nitrogen, or sulfur;
   wherein each R is as defined above and described herein.

In certain embodiments, each R⁴ is hydrogen.

In certain embodiments, each R⁴ is independently halogen. In certain embodiments, each R⁴ is independently —F. In certain embodiments, each R⁴ is independently —Cl. In certain embodiments, each R⁴ is independently —Br. In certain embodiments, each R⁴ is independently —I.

In certain embodiments, each R⁴ is independently C₁₋₃ alkyl optionally substituted with 1-3 —OR groups.

In certain embodiments, each R⁴ is independently C₁₋₃ alkyl optionally substituted with 1-3 —OH groups.

In certain embodiments, each R⁴ is independently 3-6 membered cycloalkyl.

In certain embodiments, each R⁴ is independently cyclohexyl, cyclopentyl, cyclobutyl or cyclopropyl.

In certain embodiments, two R⁴ groups on the same carbon atom are taken together to form an optionally substituted 3-6 membered saturated carbocyclic or a 3-7 membered heterocyclic ring having 1-2 heteroatoms independently selected from oxygen, nitrogen, or sulfur.

In certain embodiments, two R⁴ groups on the same carbon atom are taken together to form an optionally substituted 3-6 membered saturated carbocyclic ring. In certain embodiments, two R⁴ groups on the same carbon are taken together to form an optionally substituted cyclopropyl, cyclobutyl, cyclopentyl, cyclohexyl, or cycloheptyl ring.

In certain embodiments, two R⁴ groups on the same carbon atom are taken together to form an optionally substituted 3-7 membered heterocyclic ring having 1-2 heteroatoms independently selected from oxygen, nitrogen, or sulfur. In certain embodiments, two R⁴ groups on the same carbon atom are taken together to form an optionally substituted 3-6 membered heterocyclic ring having 1-2 heteroatoms independently selected from oxygen, nitrogen, or sulfur. In certain embodiments, two R⁴ groups on the same carbon atom are taken together to form an optionally substituted 3-5 membered heterocyclic ring having 1-2 heteroatoms independently selected from oxygen, nitrogen, or sulfur. In certain embodiments, two R⁴ groups on the same carbon atom are taken together to form an optionally substituted 3-4 membered heterocyclic ring having 1-2 heteroatoms independently selected from oxygen, nitrogen, or sulfur.

In certain embodiments, two R⁴ groups on the same carbon atom are taken together to form an optionally substituted 3-7 membered heterocyclic ring having one heteroatom independently selected from oxygen, nitrogen, or sulfur. In certain embodiments, two R⁴ groups on the same carbon atom are taken together to form an optionally substituted 3-6 membered heterocyclic ring having one heteroatom independently selected from oxygen, nitrogen, or sulfur. In certain embodiments, two R⁴ groups on the same carbon atom are taken together to form an optionally substituted 3-5 membered heterocyclic ring having one heteroatom independently selected from oxygen, nitrogen, or sulfur. In certain embodiments, two R⁴ groups on the same carbon atom are taken together to form an optionally substituted 3-4 membered heterocyclic ring having one heteroatom independently selected from oxygen, nitrogen, or sulfur.

In certain embodiments, two R⁴ groups on the same carbon are taken together to form an optionally substituted aziridinyl, azetidinyl, pyrrolidinyl, piperidinyl or piperazinyl ring.

In certain embodiments, two R⁴ groups on the same carbon are taken together to form an optionally substituted oxiranyl, oxetanyl, tetrahydrofuranyl or tetrahydro-2H-pyranyl ring.

Exemplary rings formed by two R⁴ on the same carbon atom are depicted below:

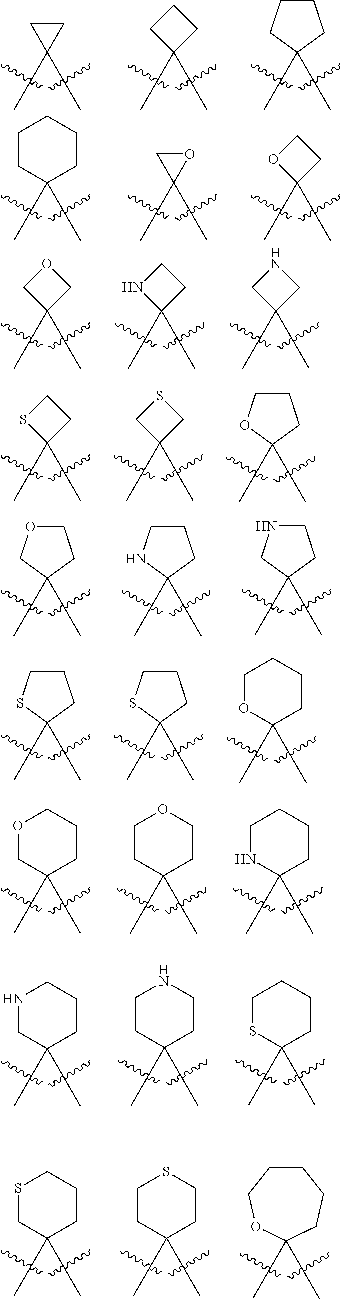

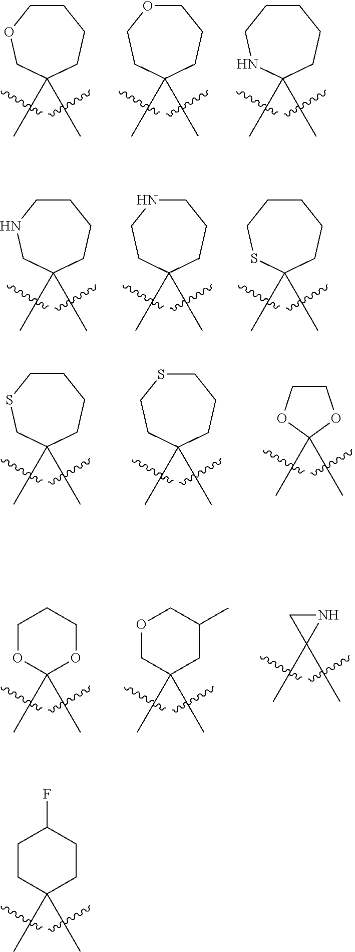

As defined generally above, each R⁵ is independently hydrogen or C₁₋₃ alkyl, or:
two R⁵ groups on the same nitrogen atom are taken together to form an optionally substituted 3-5 membered heterocyclic ring.

In certain embodiments, each R⁵ is hydrogen.

In certain embodiments, each R⁵ is independently C₁₋₃ alkyl.

In certain embodiments, two R⁵ groups on the same nitrogen atom are taken together to form an optionally substituted 3-5 membered carbocyclic ring.

Exemplary —N(R⁵)₂ groups are depicted below:

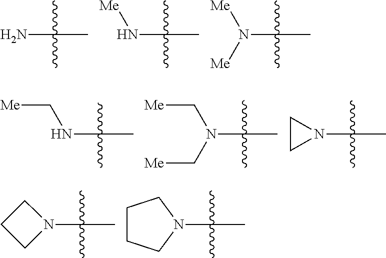

In certain embodiments, the present invention provides a compound of formula II:

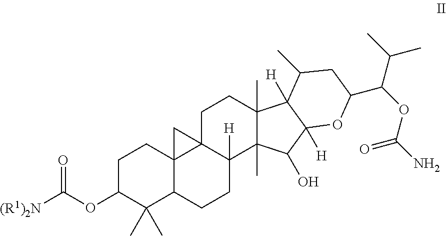

or a pharmaceutically acceptable salt thereof, wherein each R¹ is defined above and in classes and subclasses described above and herein.

In certain embodiments, the present invention provides a compound of formula II having the stereochemistry depicted in formula II-a, below:

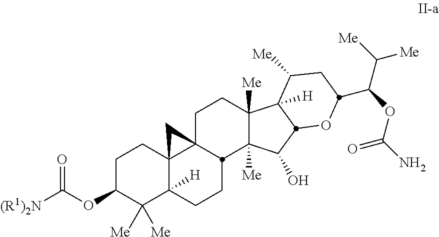

or a pharmaceutically acceptable salt thereof, wherein each R¹ is defined above and in classes and subclasses described above and herein.

In certain embodiments, the present invention provides a compound of formula III:

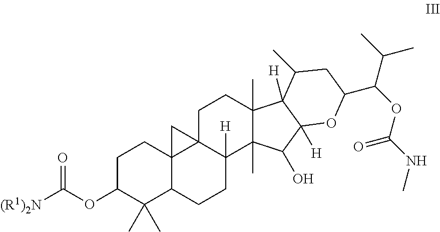

or a pharmaceutically acceptable salt thereof, wherein each R¹ is defined above and in classes and subclasses described above and herein.

In certain embodiments, the present invention provides a compound of formula III having the stereochemistry depicted in formula III-a, below:

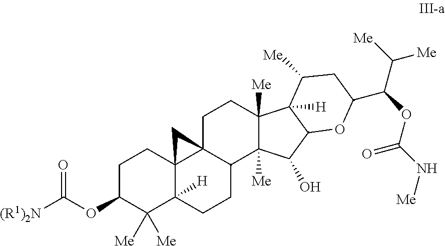

or a pharmaceutically acceptable salt thereof, wherein each R¹ is defined above and in classes and subclasses described above and herein.

In certain embodiments, the present invention provides a compound of formula IV:

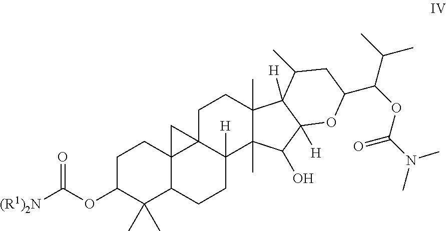

or a pharmaceutically acceptable salt thereof, wherein each R¹ is defined above and in classes and subclasses described above and herein.

In certain embodiments, the present invention provides a compound of formula IV having the stereochemistry depicted in formula IV-a, below:

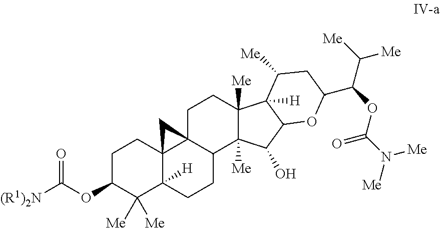

or a pharmaceutically acceptable salt thereof, wherein each R¹ is defined above and in classes and subclasses described above and herein.

In certain embodiments, the present invention provides a compound of formula V:

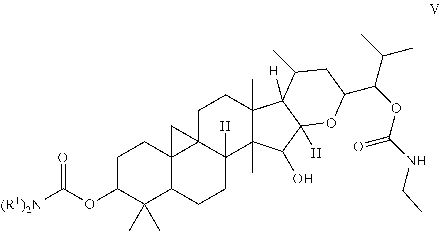

or a pharmaceutically acceptable salt thereof, wherein each R¹ is defined above and in classes and subclasses described above and herein.

In certain embodiments, the present invention provides a compound of formula V having the stereochemistry depicted in formula V-a, below:

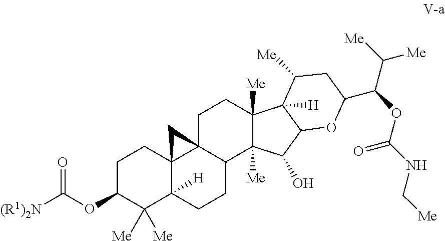

or a pharmaceutically acceptable salt thereof, wherein each R¹ is defined above and in classes and subclasses described above and herein.

In certain embodiments, the present invention provides a compound of formula VI:

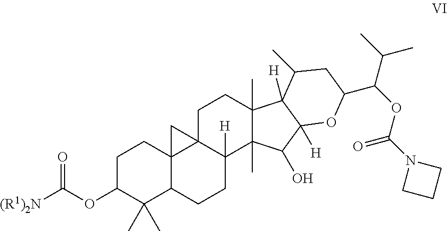

or a pharmaceutically acceptable salt thereof, wherein each R¹ is defined above and in classes and subclasses described above and herein.

In certain embodiments, the present invention provides a compound of formula VI having the stereochemistry depicted in formula VI-a, below:

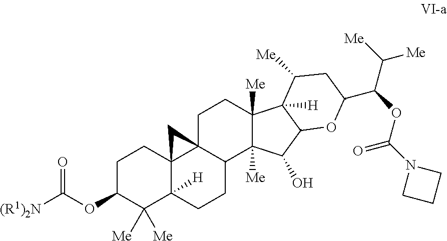

or a pharmaceutically acceptable salt thereof, wherein each R¹ is defined above and in classes and subclasses described above and herein.

In certain embodiments, the present invention provides a compound of formula VII:

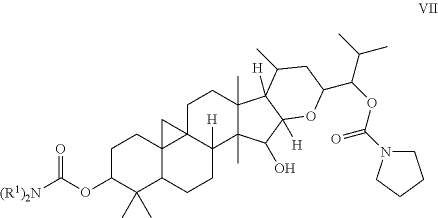

or a pharmaceutically acceptable salt thereof, wherein each R¹ is defined above and in classes and subclasses described above and herein.

In certain embodiments, the present invention provides a compound of formula VII having the stereochemistry depicted in formula VII-a, below:

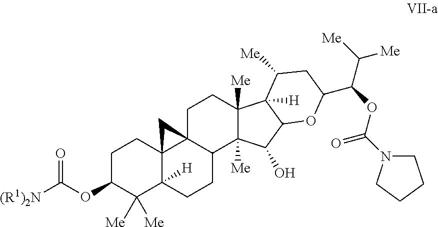

or a pharmaceutically acceptable salt thereof, wherein each R¹ is defined above and in classes and subclasses described above and herein.

As defined generally above, each R is independently hydrogen, C₁₋₄ aliphatic, or:
two R groups on the same nitrogen atom are taken together to form an optionally substituted 4-8 membered saturated or partially unsaturated ring.

In certain embodiments, each R is hydrogen.

In certain embodiments, each R is independently C₁₋₄ aliphatic. In certain embodiments, two R groups on the same nitrogen are independently C₁₋₄ aliphatic which is optionally substituted with 0-2 heteroatoms independently selected from oxygen, nitrogen, or sulfur.

In certain embodiments, two R groups on the same nitrogen atom are taken together to form an optionally substituted 4-8 membered saturated or partially unsaturated ring.

In certain embodiments, two R groups on the same nitrogen atom are taken together to form an optionally substituted 4-7 membered heterocyclic ring having 0-2 heteroatoms independently selected from oxygen, nitrogen, or sulfur in addition to the nitrogen atom where two R groups are attached.

In certain embodiments, two R groups on the same carbon are taken together to form an optionally substituted azetidinyl, pyrrolidinyl, piperidinyl or piperazinyl ring.

In certain embodiments, two R groups on the same carbon are taken together to form an azetidinyl, pyrrolidinyl, piperidinyl or piperazinyl ring optionally substituted with one or more —F groups.

In certain embodiments, two R groups on the same carbon are taken together to form an azetidinyl, pyrrolidinyl, piperidinyl or piperazinyl ring optionally substituted with one or more optionally substituted C₁₋₃ alkyl groups.

In certain embodiments, two R groups on the same carbon are taken together to form an azetidinyl, pyrrolidinyl, piperidinyl or piperazinyl ring optionally substituted with one or more methoxy groups.

Exemplary —N(R)₂ groups are depicted below:

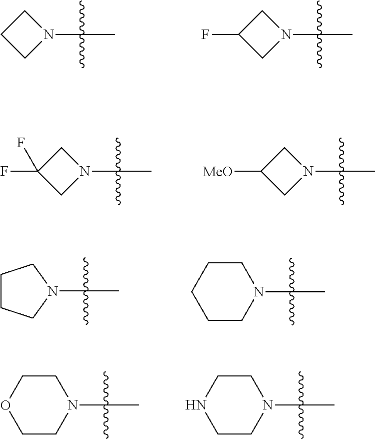

Exemplary compounds of formula I are set forth in Table 1, below.

TABLE 1
Exemplary Compounds

| | |
|---|---|
| 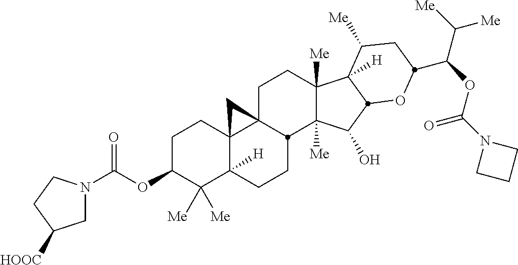 | I-1 |
| 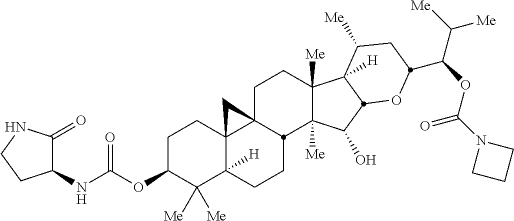 | I-2 |
| 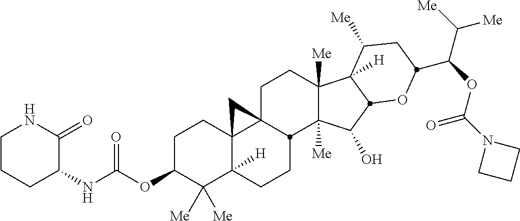 | I-3 |
| 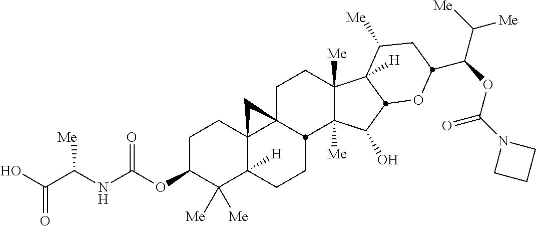 | I-4 |
| 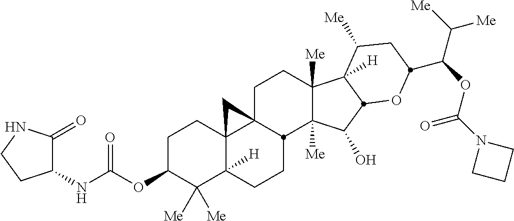 | I-5 |
| 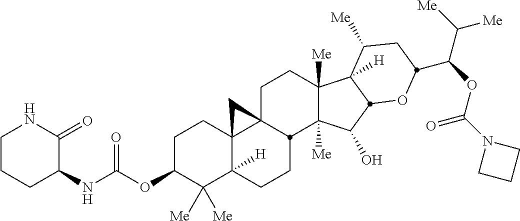 | I-6 |
| 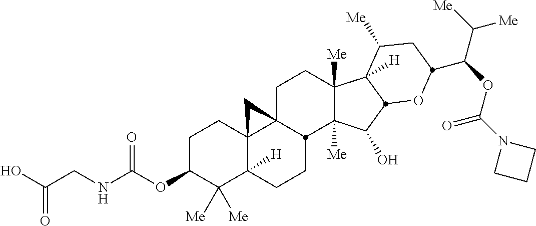 | I-7 |
| 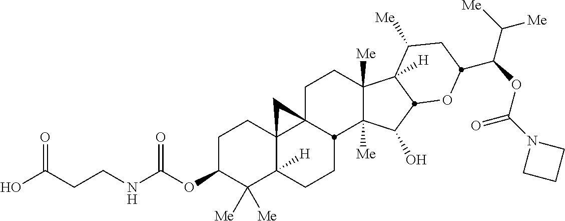 | I-8 |
| 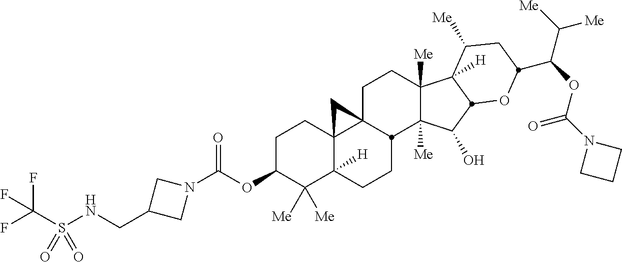 | I-9 |
| 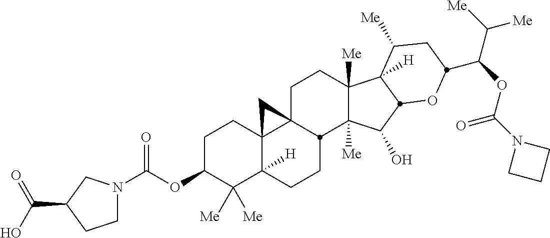 | I-10 |
| 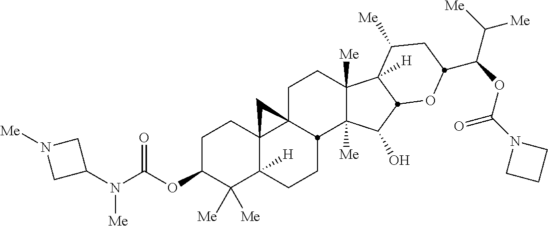 | I-11 |
| 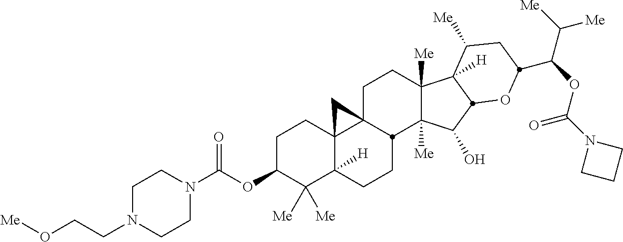 | I-12 |
| 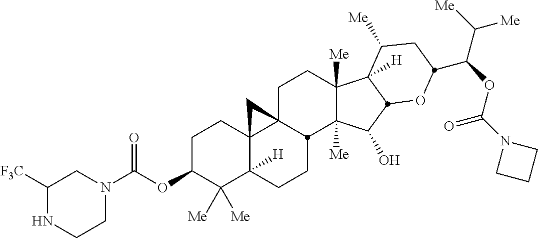 | I-13 |
| 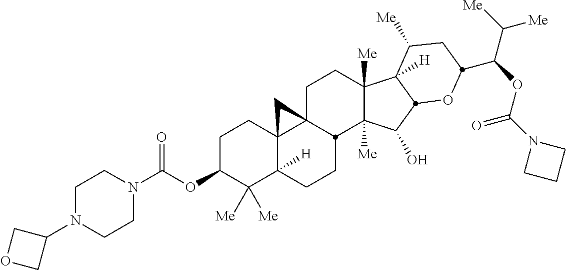 | I-14 |
| 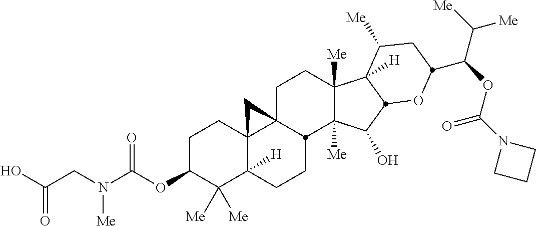 | I-15 |
| 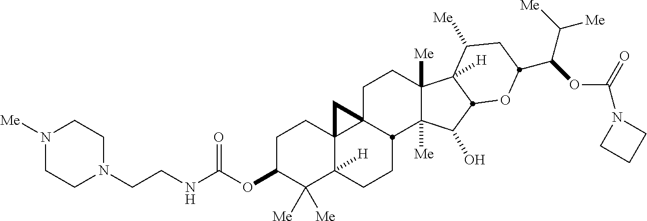 | I-16 |
| 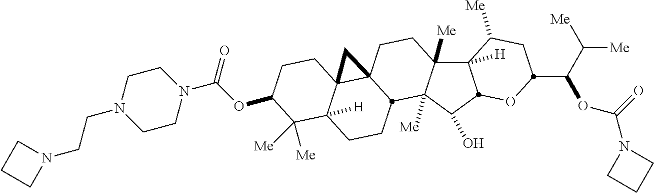 | I-17 |
| 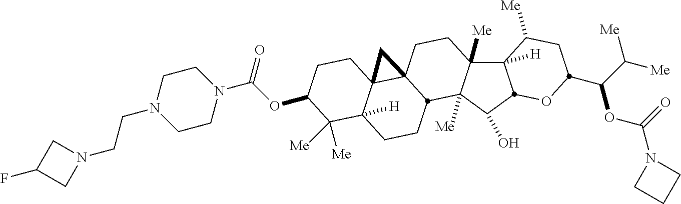 | I-18 |
| 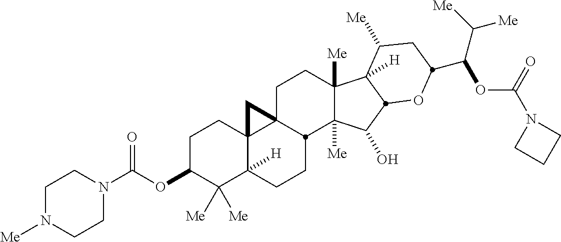 | I-19 |
| 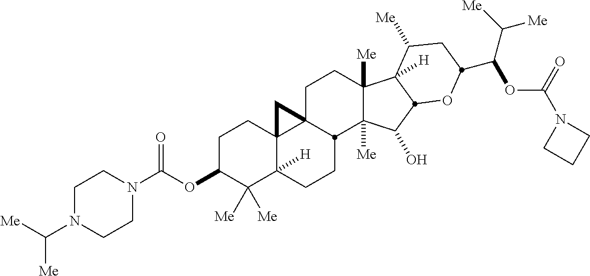 | I-20 |
| 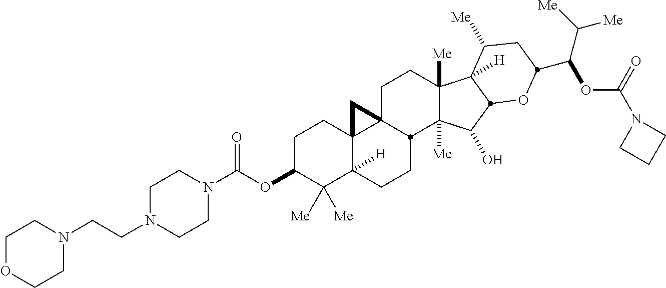 | I-21 |
| 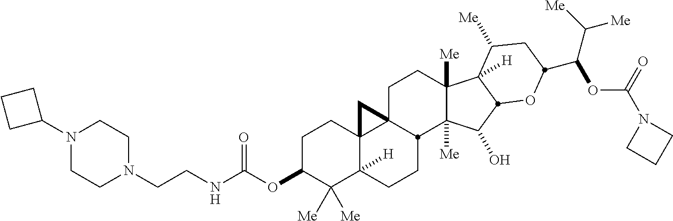 | I-22 |
| 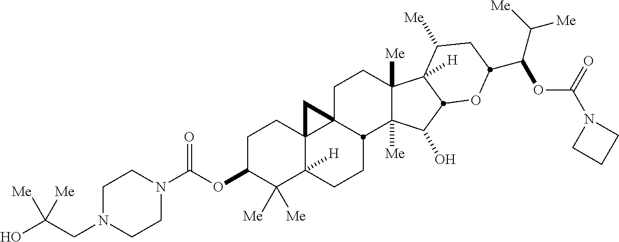 | I-23 |
| 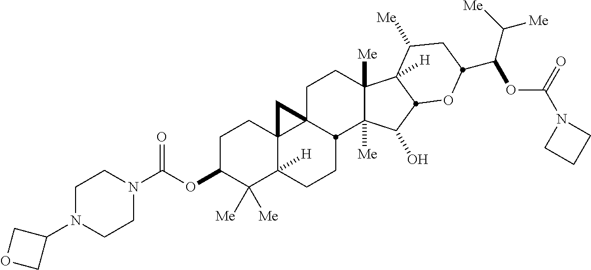 | I-24 |
| 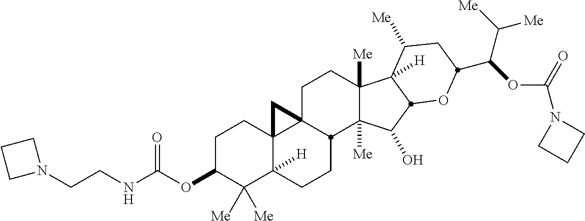 | I-25 |
| 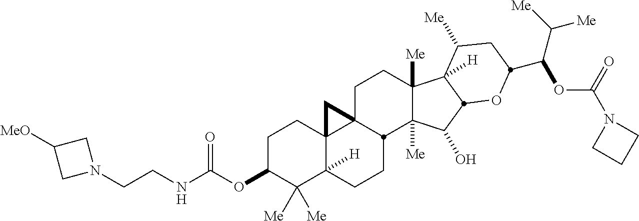 | I-26 |
| 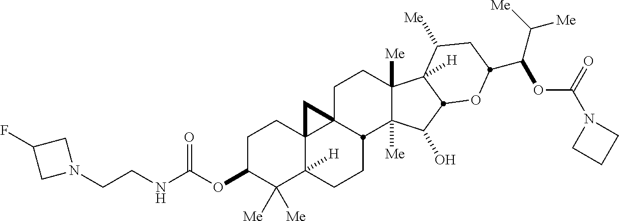 | I-27 |
| 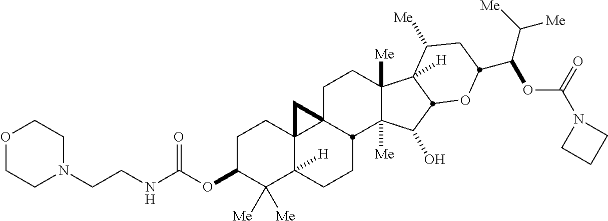 | I-28 |
| 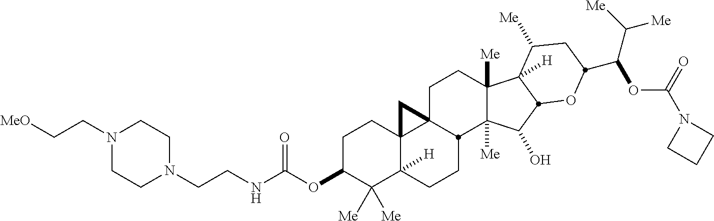 | I-29 |
| 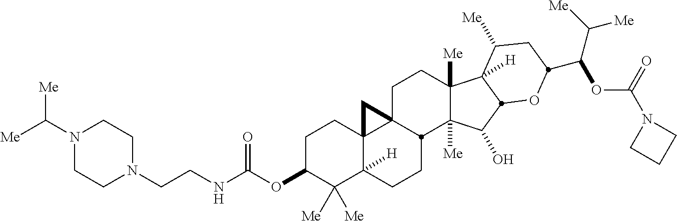 | I-30 |
| 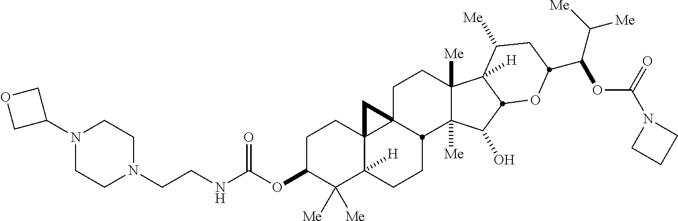 | I-31 |
| 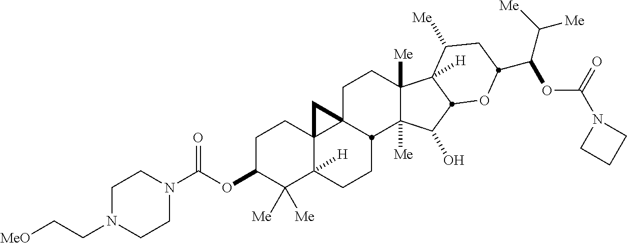 | I-32 |
| 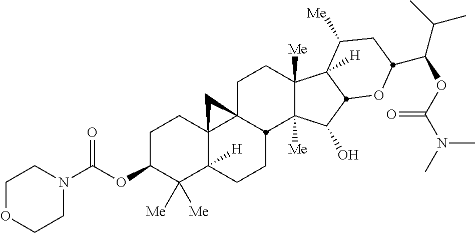 | I-33 |
| 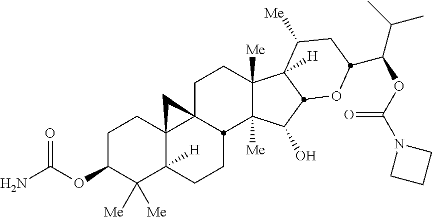 | I-34 |
| 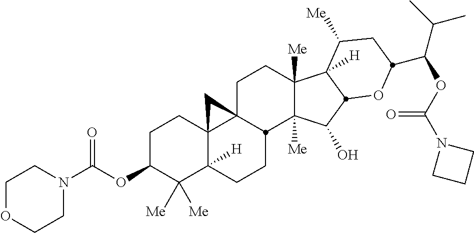 | I-35 |
| 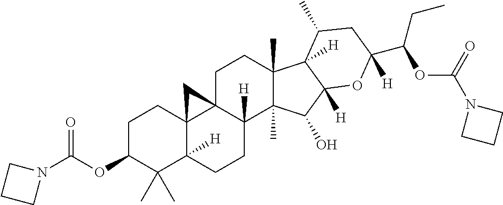 | I-36 |
| 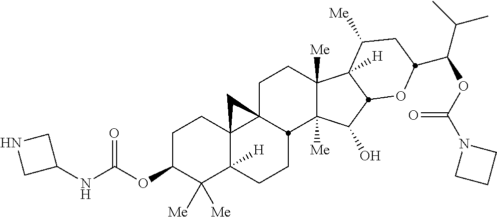 | I-37 |
| 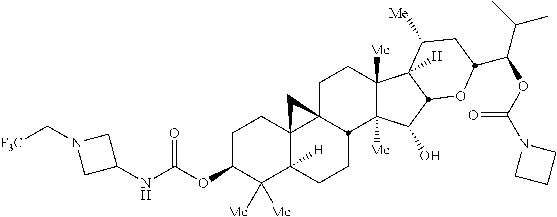 | I-38 |
| 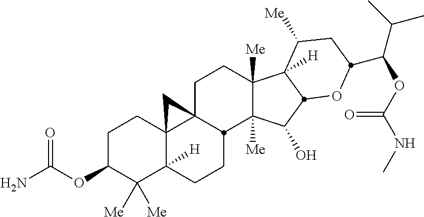 | I-39 |
| 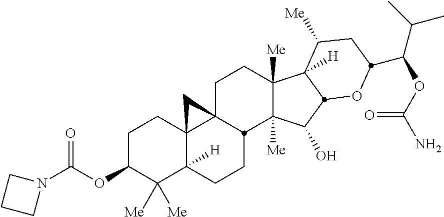 | I-40 |
| 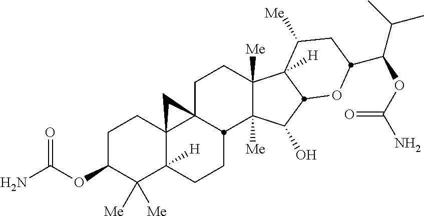 | I-41 |
| 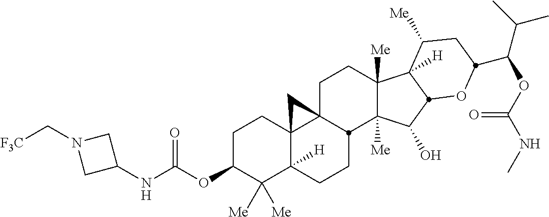 | I-42 |
| 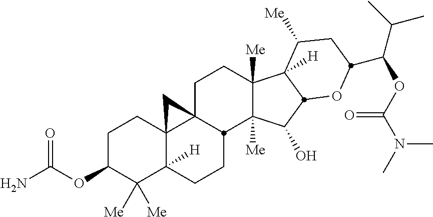 | I-43 |
| 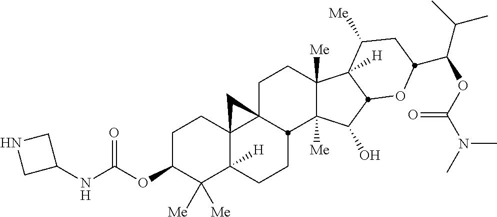 | I-44 |
| 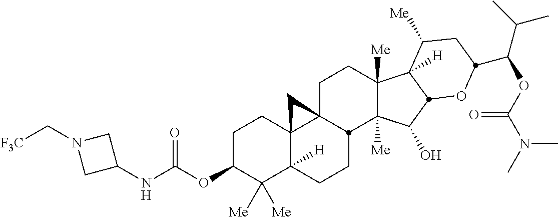 | I-45 |
| 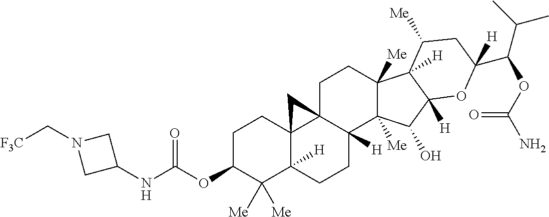 | I-46 |
| 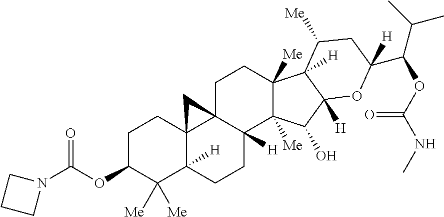 | I-47 |
| 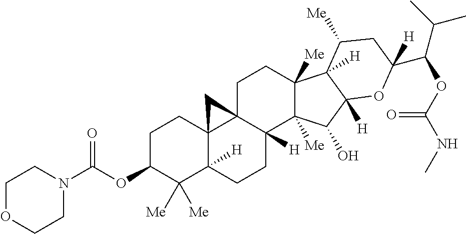 | I-48 |
| 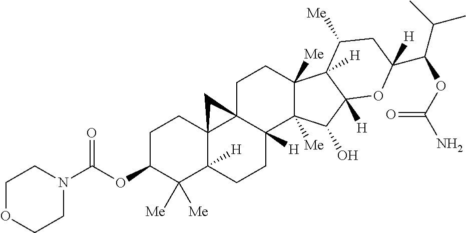 | I-49 |
| 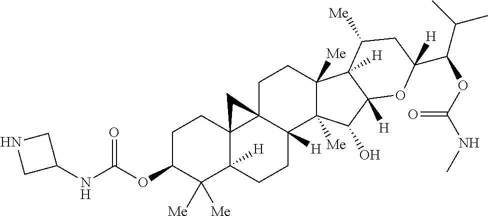 | I-50 |
| 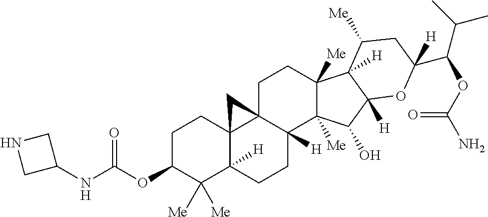 | I-51 |
| 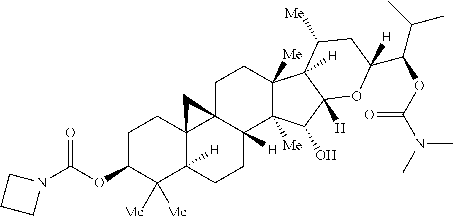 | I-52 |
| 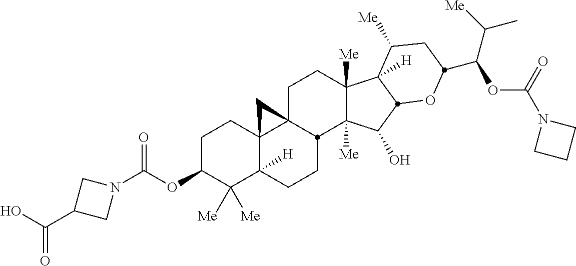 | I-53 |
| 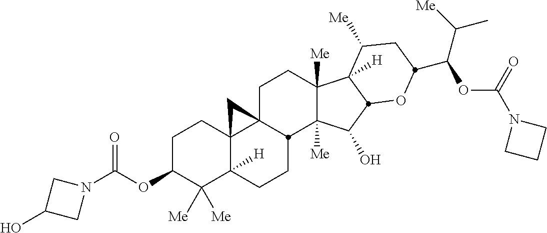 | I-54 |
| 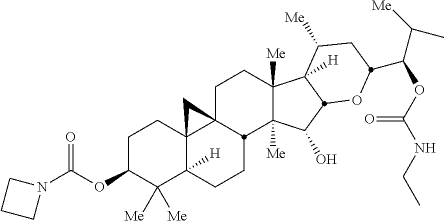 | I-55 |
| 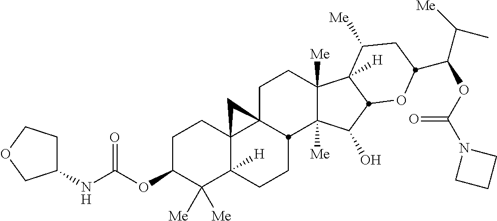 | I-56 |
| 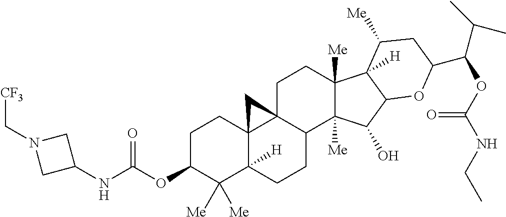 | I-57 |
| 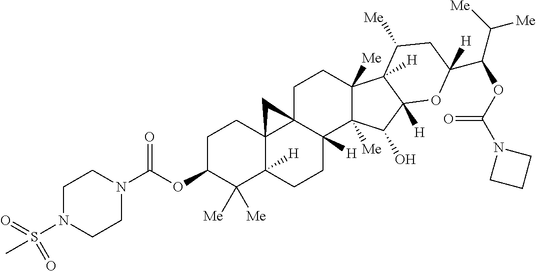 | I-58 |
| 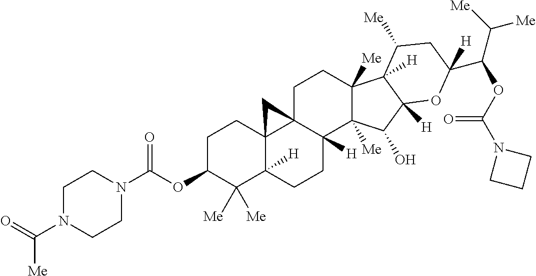 | I-59 |
| 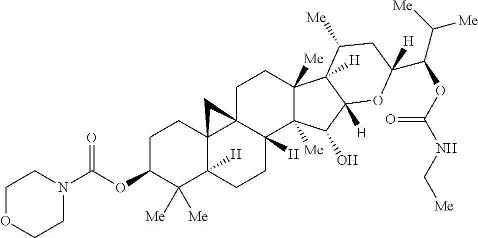 | I-60 |
| 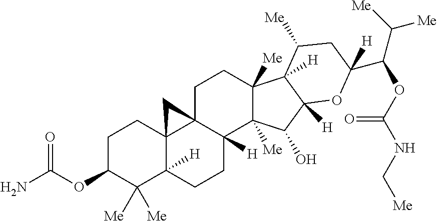 | I-61 |
| 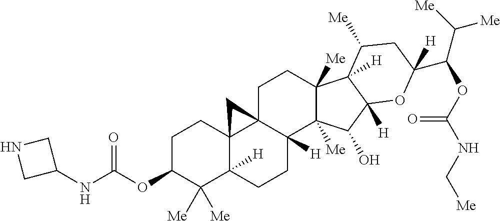 | I-62 |
| 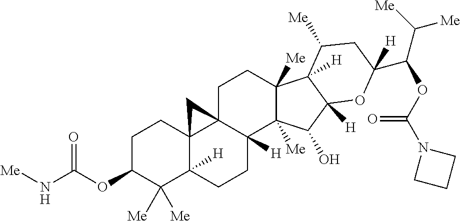 | I-63 |
| 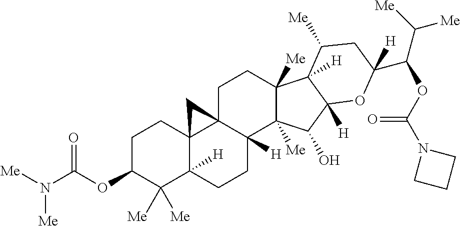 | I-64 |
| 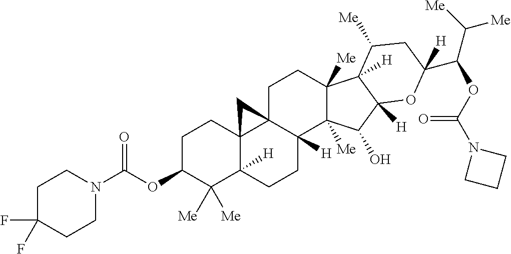 | I-65 |
| 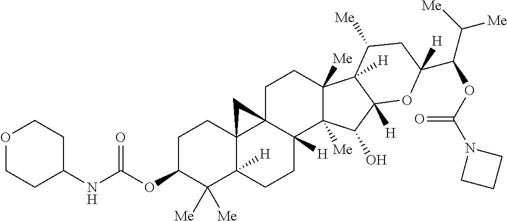 | I-66 |
| 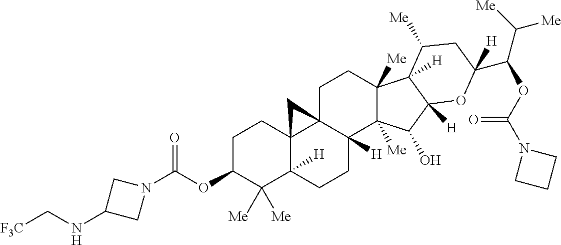 | I-67 |
| 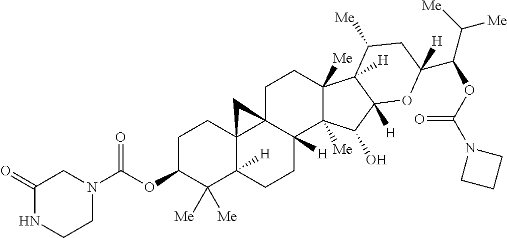 | I-68 |
| 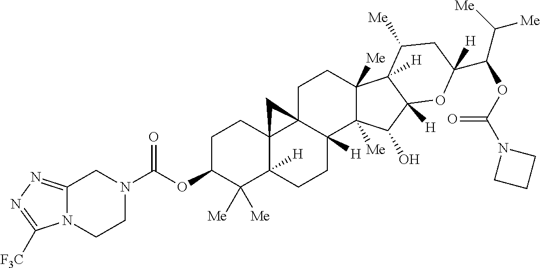 | I-69 |
| 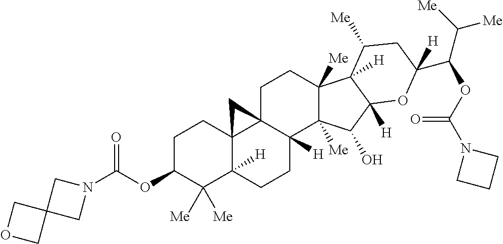 | I-70 |
| 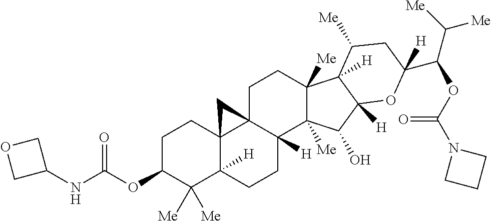 | I-71 |
| 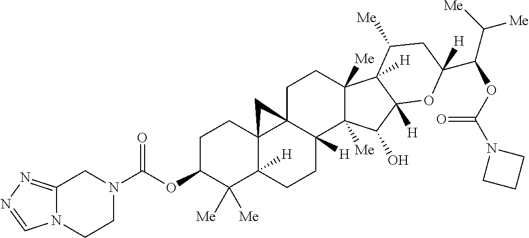 | I-72 |
| 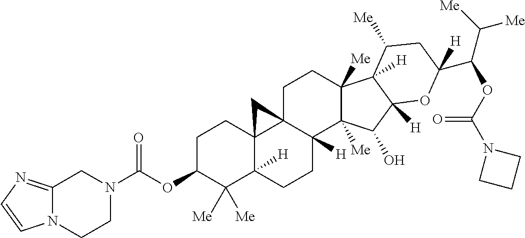 | I-73 |
| 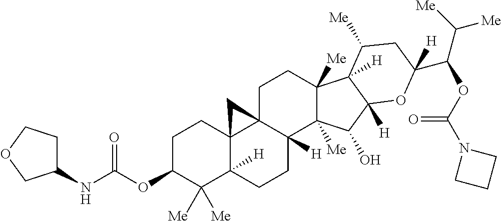 | I-74 |
| 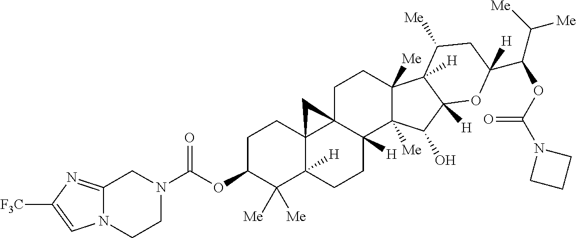 | I-75 |
| 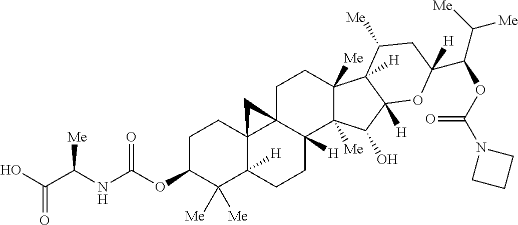 | I-76 |
| 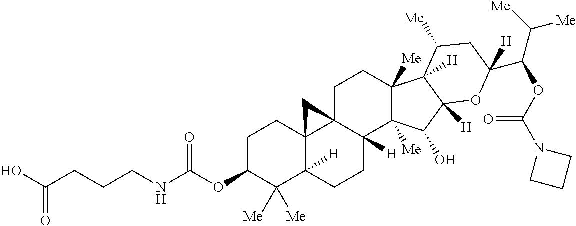 | I-77 |
| 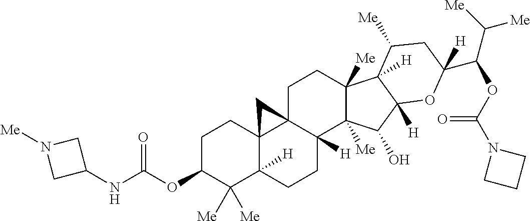 | I-78 |
| 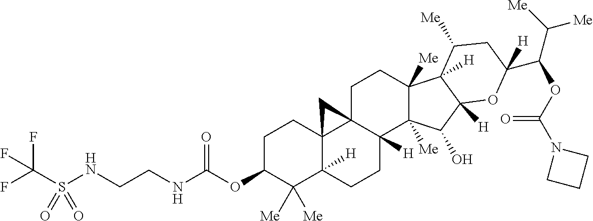 | I-79 |

### 4. General Methods of Providing the Present Compounds

The compounds of this invention may be prepared or isolated in general by synthetic and/or semi-synthetic methods known to those skilled in the art for analogous compounds and by methods described in detail in the Examples, herein. Methods and intermediates of the present invention are useful for preparing compounds as described in, e.g. U.S. patent application Ser. No. 13/040,166, filed Mar. 3, 2011, in the name of Bronk et al., the entirety of which is incorporated herein by reference.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3rd edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like. In certain embodiments, the amino protecting group of the R¹⁰ moiety is phthalimido. In still other embodiments, the amino protecting group of the R¹⁰ moiety is a tert-butyloxycarbonyl (BOC) group. In certain embodiments, the amino protecting group is a sulphone (SO₂R).

Isolation of Material from Biomass

Certain compounds used in methods of the present invention are isolated from black cohosh root, also known as cimicifuga racemosa or actaea racemosa. Commercial extracts, powders, and capsules of black cohosh root are available for treating a variety of menopausal and gynecological disorders. However, it has been surprisingly found that certain compounds present in black cohosh root are useful for modulating and/or inhibiting amyloid-beta peptide production. In particular, certain compounds have been isolated from black cohosh root and identified, wherein these compounds are useful as synthetic precursors en route to compounds useful for modulating and/or inhibiting amyloid-beta peptide production, and in particular amyloid-beta peptide (1-42). These compounds may be isolated and utilized in a form substantially free of other compounds normally found in the root.

In some embodiments, methods of the present invention for use in preparing a compound of formula II use compounds found in extracts of black cohosh and related cimicifuga species, whether from roots and rhizome or aerial parts of these plants. One of ordinary skill in the art will recognize that synthetic precursors may be obtained from one or more cimicifuga species including, but not limited to, Cimicifuga racemosa, Cimicifuga dahurica, Cimicifuga foetida, Cimicifuga heracleifolia, Cimicifuga japonica, Cimicifuga acerina, Cimicifuga acerima, Cimicifuga simplex, and Cimicifuga elata, Cimicifuga calthaefolia, Cimicifuga frigida, Cimicifuga laciniata, Cimicifuga mairei, Cimicifuga rubifolia, Cimicifuga americana, Cimicifuga biternata, and Cimicifuga bifida or a variety thereof. This may be accomplished either by chemical or biological transformation of an isolated compound or an extract fraction or mixture of compounds. Chemical transformation may be accomplished by, but not limited to, manipulation of temperature, pH, and/or treatment with various solvents and/or reagents. Biological transformation may be accomplished by, but not limited to, treatment of an isolated compound or an extract fraction or mixture of compounds with plant tissue, plant tissue extracts, other microbiological organisms or an isolated enzyme from any organism.

In some embodiments, a precursor compound is extracted from a sample of biomass to provide a compound of formula A, as depicted in Scheme I below.

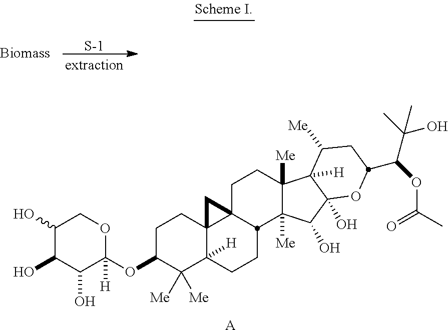

The term “biomass,” as used herein, refers to roots, rhizomes and/or aerial parts of the cimicifuga species of plant, as described above and herein.

In some embodiments, the process of obtaining a compound of formula A from biomass comprises a step of pre-treating the biomass. In some embodiments, the step of pretreating comprises a step of drying. In certain embodiments, the step of drying comprises use of one or more suitable methods for providing biomass of a desired level of dryness. For instance, in some embodiments the biomass is dried using vacuum. In some embodiments, the biomass is dried using heat. In some embodiments, the biomass is dried using a spray dryer or drum dryer. In some embodiments, the biomass is dried using two or more of the above methods.

In some embodiments, the step of pretreating comprises a step of grinding. In certain embodiments, the step of grinding comprises passing the sample of biomass through a chipper or grinding mill for an amount of time suitable to provide biomass of a desired particle size. In some embodiments, the biomass is dried prior to being ground to a suitable particle size.

In some embodiments, a suitable particle size ranges from about 0.1 mm³ to about 1.0 mm³. In some embodiments, a suitable particle size ranges from about 0.2 mm³ to about 1.0 mm³. In some embodiments, a suitable particle size ranges from about 0.3 mm³ to about 1.0 mm³. In some embodiments, a suitable particle size ranges from about 0.4 mm³ to about 1.0 mm³. In some embodiments, a suitable particle size ranges from about 0.5 mm³ to about 1.0 mm³. In some embodiments, a suitable particle size ranges from about 0.6 mm³ to about 1.0 mm³. In some embodiments, a suitable particle size ranges from about 0.7 mm³ to about 1.0 mm³. In some embodiments, a suitable particle size ranges from about 0.8 mm³ to about 1.0 mm³. In some embodiments, a suitable particle size ranges from about 0.9 mm³ to about 1.0 mm³.

In some embodiments, a suitable particle size ranges from about 0.1 mm³ to about 0.9 mm³. In some embodiments, a suitable particle size ranges from about 0.1 mm³ to about 0.8 mm³. In some embodiments, a suitable particle size ranges from about 0.1 mm³ to about 0.7 mm³. In some embodiments, a suitable particle size ranges from about 0.1 mm³ to about 0.6 mm³. In some embodiments, a suitable particle size ranges from about 0.1 mm³ to about 0.5 mm³. In some embodiments, a suitable particle size ranges from about 0.1 mm³ to about 0.4 mm³. In some embodiments, a suitable particle size ranges from about 0.1 mm³ to about 0.3 mm³. In some embodiments, a suitable particle size ranges from about 0.1 mm³ to about 0.2 mm³.

In some embodiments, biomass is dried and ground prior to being extracted. The term “extraction,” as used herein, refers to the general process of obtaining a compound of formula A comprising a step of exposing biomass to one or more suitable solvents under suitable conditions for a suitable amount of time in order to extract a compound of formula A from the biomass. In some embodiments, extraction comprises agitating and heating a slurry comprised of biomass and one or more suitable solvents. In certain embodiments, the one or more suitable solvents comprise one or more alcohols, and optionally water. Suitable alcohols include, but are not limited to, methanol, ethanol, isopropanol, and the like. In certain embodiments, the alcohol is methanol. In certain embodiments, the alcohol is ethanol. In some embodiments, the slurry is heated to a temperature of about 25° C., 30° C., 35° C., 40° C., 45° C., 50° C., 55° C., 60° C., 65° C., and 70° C. In some embodiments, an elevated temperature is a temperature of greater than about 70° C. In certain embodiments, the slurry is heated to about 50° C. In certain embodiments, the slurry is kept at ambient temperature.

In some embodiments, the biomass is exposed to one or more suitable solvents under suitable conditions for an amount of time ranging from about 0.1 h to about 48 h. In some embodiments, the amount of time ranges from about 0.1 h to about 36 h. In some embodiments, the amount of time ranges from about 0.1 h to about 24 h. In some embodiments, the amount of time ranges from about 0.5 h to about 24 h. In some embodiments, the amount of time ranges from about 1 h to about 24 h. In some embodiments, the amount of time ranges from about 2 h to about 24 h. In some embodiments, the amount of time ranges from about 2 h to about 22 h. In some embodiments, the amount of time ranges from about 2 h to about 20 h. In some embodiments, the amount of time ranges from about 2 h to about 4 h. In some embodiments, the amount of time ranges from about 20 h to about 24 h. In some embodiments, the amount of time is about 2 h. In some embodiments, the amount of time is about 22 h.

In some embodiments, once the slurry of biomass is heated and/or agitated for a suitable amount of time, the slurry is filtered through e.g., Celite, and concentrated down to the crude extract. In certain embodiments, the crude extract is further treated with an aqueous salt solution such as, e.g., 5% aqueous KCl, and cooled to a temperature of about 2° C. to about 10° C. Exemplary other salts for use in an aqueous salt solution include, but are not limited to, (NH₄)SO₄, K₂SO₄, NaCl, etc. In some embodiments, the aqueous salt solution has a concentration ranging from about 1% to about 50%. In some embodiments, the aqueous salt solution has a concentration ranging from about 3% to about 30%. In some embodiments, the aqueous salt solution has a concentration ranging from about 5% to about 10%. In some embodiments, the aqueous salt solution has a concentration ranging from about 10% to about 20%. In some embodiments, the aqueous salt solution has a concentration ranging from about 20% to about 30%. In certain embodiments, the crude extract is cooled to a temperature of about 2° C. to about 6° C. In certain embodiments, the crude extract is cooled to a temperature of about 4° C. In some embodiments, the crude extract is cooled for about 1, 2, 3, 4, or 5 h. In certain embodiments, the crude extract is cooled for about 2 h. In some embodiments, the crude extract is cooled for more than about 5 h. In certain embodiments, the crude extract is cooled for about 5 h to about 10 h. In certain embodiments, the crude extract is cooled for about 10 h to about 15 h. In certain embodiments, the crude extract is cooled for about 15 h to about 20 h. In certain embodiments, the crude extract is cooled for about 20 h to about 25 h. In some embodiments, after the crude extract is cooled for an appropriate amount of time, the slurry is centrifuged and the resulting solids are collected and dried using any one or more methods known in the art.

In some embodiments, step S-1 provides compound A in about 3-15% purity.

In some embodiments, the present invention provides a method for obtaining a compound of formula A. In certain embodiments, the present invention provides a method for obtaining a compound of formula A from biomass comprising the step of contacting the biomass with one or more suitable solvents under suitable conditions for a suitable amount of time to obtain a compound of formula A.

### General Method for Preparing Compounds of Formula I

In some embodiments, compound A serves as starting material in the synthesis of a compound of formula I, as illustrated in Scheme II, below.

As depicted in step S-2 of Scheme II, a compound of formula A is converted by dehydration to provide carbonyl compound B, which is subsequently reduced at the ring carbonyl, then deprotected to provide intermediate C.

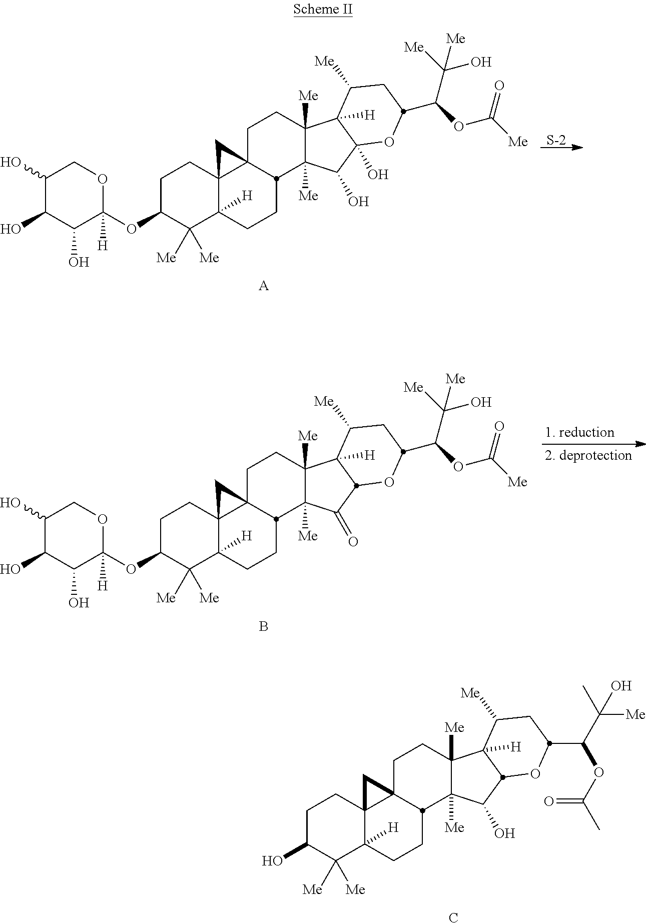

In some embodiments, the oxygen protection of C in step S-4 provides D, wherein each variable is defined above and described in classes and subclasses above and herein, as illustrated in Scheme III, below. In step S-5, the tertiary free hydroxyl group of formula D is removed to provide olefin E, wherein each variable is defined above and described in classes and subclasses above and herein, followed by deacylation in step S-6 to provide F, wherein each variable is defined above and described in classes and subclasses above and herein.

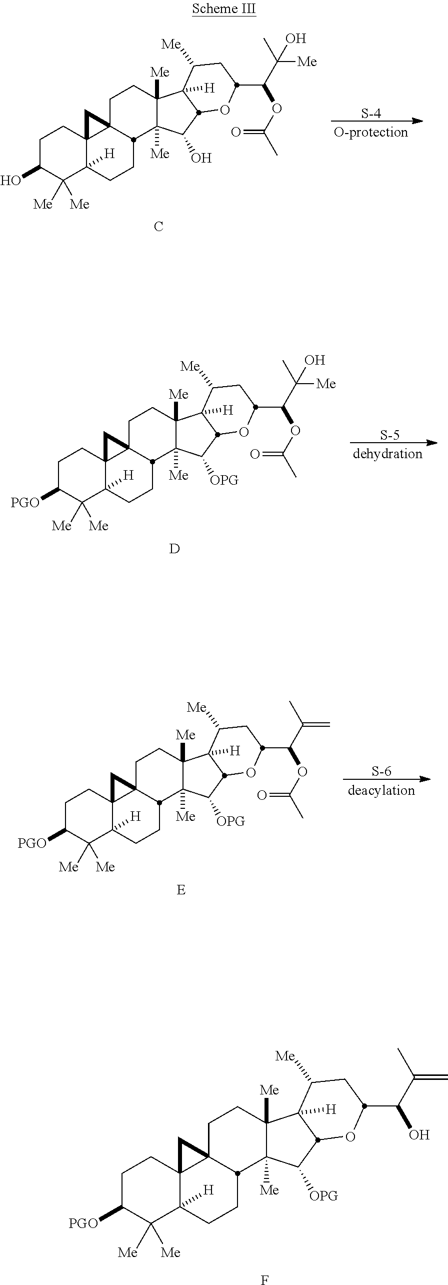

In some embodiments, intermediate F in step S-7 is coupled to provide carbamate G, wherein each variable is defined above and described in classes and subclasses above and herein, as illustrated in Scheme IV, below. In step S-8, the olefin is reduced and the oxygen protecting groups are removed by hydrogenation to provide intermediate H. Step S-9 provides installation of another carbamate group to provide the compounds of formula I, wherein each variable is defined above and described in classes and subclasses above and herein.

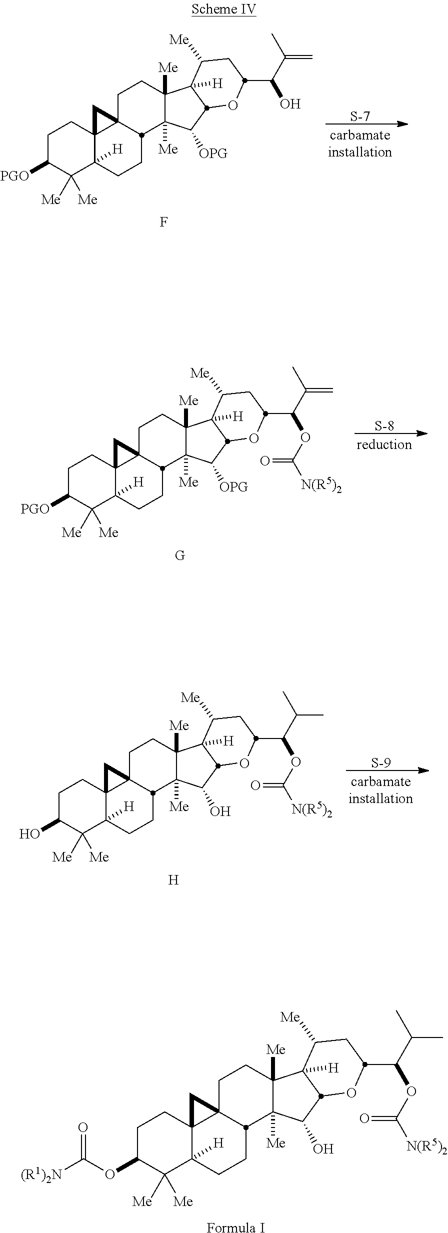

### V. Uses, Formulation and Administration

### Pharmaceutically Acceptable Compositions

According to another aspect of the present invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable salt thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or a pharmaceutically active metabolite or residue thereof. As used herein, the term “pharmaceutically active metabolite or residue thereof” means that a metabolite or residue thereof is also a pharmaceutically active compound in accordance with the present invention.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some cases, compounds of the present invention may contain one or more acidic functional groups and, thus, may be capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. See, for example, Berge et al., supra.

The compositions of the present invention may additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compositions provided by the present invention can be employed in combination therapies, meaning that the present compositions can be administered concurrently with, prior to, or subsequent to, one or more other desired therapeutic agents or medical procedures. The particular combination of therapies (therapeutic agents or procedures) to employ in a combination regimen will take into account compatibility of the desired therapeutic agents and/or procedures and the desired therapeutic effect to be achieved. It will also be appreciated that the therapies employed may achieve a desired effect for the same disorder (for example, a compound described herein may be administered concurrently with another therapeutic agent used to treat the same disorder), or they may achieve different effects (e.g., control of any adverse effects).

For example, known agents useful for treating neurodegenerative disorders may be combined with the compositions of this invention to treat neurodegenerative disorders, such as Alzheimer's disease. Examples of such known agents useful for treating neurodegenerative disorders include, but are not limited to, treatments for Alzheimer's disease such as acetylcholinesterase inhibitors, including donepezil, Exelon® and others; memantine (and related compounds as NMDA inhibitors), treatments for Parkinson's disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; riluzole, and anti-Parkinsonian agents. For a more comprehensive discussion of updated therapies useful for treating neurodegenerative disorders, see, a list of the FDA approved drugs at http://www.fda.gov, and The Merck Manual, Seventeenth Ed. 1999, the entire contents of which are hereby incorporated by reference.

Additional examples of such known agents useful for treating neurodegenerative disorders include, but are not limited to, beta-secretase inhibitors/modulators, gamma-secretase inhibitors/modulators, HMG-CoA reductase inhibitors, NSAID's including ibuprofen, vitamin E, anti-amyloid antibodies, including humanized monoclonal antibodies, inhibitors/modulators of tau phosphorylation (such as GSK3 or CDK inhibitors/modulators) and/or aggregation, CB-1 receptor antagonists or CB-1 receptor inverse agonists, antibiotics such as doxycycline and rifampin, N-methyl-D-aspartate (NMDA) receptor antagonists, such as mematine, cholinesterase inhibitors such as galantamine, rivastigmnine, donepezil and tacrine, growth hormone secretagogues such as ibutamoren, ibutamoren mesylate and capromorelin, histamine H₃ antagonists, AMPA agonists, PDE-IV, -V, -VII, -VIII, and -IX inhibitors, GABA_{A} inverse agonists, and neuronal nicotinic agonists and partial agonists, serotonin receptor antagonists.

In other embodiments, the compounds of the present invention are combined with other agents useful for treating neurodegenerative disorders, such as Alzheimer's disease, wherein such agents include beta-secretase inhibitors/modulators, gamma-secretase inhibitors/modulators, anti-amyloid antibodies, including humanized monoclonal antibodies aggregation inhibitors, metal chelators, antioxidants, and neuroprotectants and inhibitors/modulators of tau phosphorylation (such as GSK3 or CDK inhibitors/modulators) and/or aggregation.

In some embodiments, compounds of the present invention are combined with gamma secretase modulators. In some embodiments, compounds of the present invention are gamma secretase modulators combined with gamma secretase modulators. Exemplary such gamma secretase modulators include, inter alia, certain NSAIDs and their analogs (see WO01/78721 and US 2002/0128319 and Weggen et al., Nature, 414 (2001) 212-16; Morihara et al., J. Neurochem., 83 (2002), 1009-12; and Takahashi et al., J. Biol. Chem., 278 (2003), 18644-70).

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a provided compound, an additional therapeutic agent, and a pharmaceutically acceptable carrier, adjuvant, or vehicle.

Other examples of agents the compounds of this invention may also be combined with include, without limitation: treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions of this invention will be no more than the amount that would normally be administered in a composition comprising that therapeutic agent as the only active agent. In certain embodiments, the amount of additional therapeutic agent in the present compositions will range from about 50% to 100% of the amount normally present in a composition comprising that agent as the only therapeutically active agent.

In an alternate embodiment, the methods of this invention that utilize compositions that do not contain an additional therapeutic agent, comprise the additional step of separately administering to said patient an additional therapeutic agent. When these additional therapeutic agents are administered separately they may be administered to the patient prior to, sequentially with or following administration of the compositions of this invention.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disorder being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with one or more inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with one or more inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the present invention provides a composition containing a provided compound in an amount of about 1 weight percent to about 99 weight percent. In other embodiments, the composition contains a provided compound wherein the composition contains no more than about 10.0 area percent HPLC of other components of black cohosh root relative to the total area of the HPLC chromatogram. In other embodiments, the composition containing a provided compound contains no more than about 8.0 area percent HPLC of other components of black cohosh root relative to the total area of the HPLC chromatogram, and in still other embodiments, no more than about 3 area percent.

### Uses of Compounds and Pharmaceutically Acceptable Compositions

Alzheimer's Disease (AD) is believed to result from the deposition of quantities of a peptide, amyloid-beta (“A-beta”), within the brain. This peptide is produced by enzymatic cleavage of amyloid protein precursor (“APP”) protein. The C-terminus of A-beta is generated by an enzyme termed gamma-secretase. Cleavage occurs at more than one site on APP producing different length A-beta peptides, some of which are more prone to deposition, such as A-beta 42. It is believed that aberrant production A-beta 42 in the brain leads to AD.

A-beta, a 37-43 amino acid peptide derived by proteolytic cleavage of the amyloid precursor protein (APP), is the major component of amyloid plaques. APP is expressed and constitutively catabolized in most cells. APP has a short half-life and is metabolized rapidly down two pathways. In one pathway, cleavage by an enzyme known as alpha-secretase occurs while APP is still in the trans-Golgi secretory compartment. This cleavage by alpha-secretase occurs within the A-beta portion of APP, thus precluding the formation of A-beta.

In contrast to this non-amyloidogenic pathway involving alpha-secretase described above, proteolytic processing of APP by beta-secretase exposes the N-terminus of A-beta, which after gamma-secretase cleavage at the variable C-terminus, liberates A-beta. Peptides of 40 or 42 amino acids in length (A-beta 1-40 and A-beta 1-42, respectively) predominate among the C-termini generated by gamma-secretase, however, a recent report suggests 1-38 is a dominant species in cerebrospinal fluid. A-beta 1-42 is more prone to aggregation than A-beta 1-40, the major component of amyloid plaque, and its production is closely associated with the development of Alzheimer's disease. The bond cleaved by gamma-secretase appears to be situated within the transmembrane domain of APP. In the amyloidogenic pathway, APP is cleaved by beta-secretase to liberate sAPP-beta and CTF-beta, which CTF-beta is then cleaved by gamma-secretase to liberate the harmful A-beta peptide.

While abundant evidence suggests that extracellular accumulation and deposition of A-beta is a central event in the etiology of AD, recent studies have also proposed that increased intracellular accumulation of A-beta or amyloid containing C-terminal fragments may play a role in the pathophysiology of AD. For example, over-expression of APP harboring mutations which cause familial Alzheimer's disease (AD) results in the increased intracellular accumulation of CTF-beta in neuronal cultures and A-beta 42 in HEK 293 cells.

A-beta 42 is the 42 amino acid long form of A-beta that is believed to be more potent in forming amyloid plaques than the shorter forms of A-beta. Moreover, evidence suggests that intra- and extracellular A-beta are formed in distinct cellular pools in hippocampal neurons and that a common feature associated with two types of familial AD mutations in APP (“Swedish” and “London”) is an increased intracellular accumulation of A-beta 42.

Without wishing to be bound by theory, it is believed that of importance in this A-beta-producing pathway is the position of the gamma-secretase cleavage. If the gamma-secretase proteolytic cut is at residue or before 711-712, shorter A-beta. (A-beta 40 or shorter) is the result; if it is a proteolytic cut after residue 713, long A-beta (A-beta 42) is the result. Thus, the gamma secretase process is central to the production of A-beta peptide of 40 or 42 amino acids in length (A-beta 40 and A-beta 42, respectively). For a review that discusses APP and its processing, see Selkoe, 1998, Trends Cell. Biol. 8:447-453; Selkoe, 1994, Ann. Rev. Cell Biol. 10:373-403. See also, Esch et al., 1994, Science 248:1122.

Cleavage of APP can be detected in a number of convenient manners, including the detection of polypeptide or peptide fragments produced by proteolysis. Such fragments can be detected by any convenient means, such as by antibody binding. Another convenient method for detecting proteolytic cleavage is through the use of a chromogenic beta secretase substrate whereby cleavage of the substrate releases a chromogen, e.g., a colored or fluorescent, product. More detailed analyses can be performed including mass spectroscopy.

Much interest has focused on the possibility of inhibiting the development of amyloid plaques as a means of preventing or ameliorating the symptoms of Alzheimer's disease. To that end, a promising strategy is to inhibit the activity of beta- and/or gamma-secretase, the two enzymes that together are responsible for producing A-beta. This strategy is attractive because, if amyloid plaque formation as a result of A-beta deposition is a cause of Alzheimer's disease, then inhibiting the activity of one or both of the two secretases would intervene in the disease process at an early stage, before late-stage events such as inflammation or apoptosis occur.

Modulators of gamma-secretase may function in a variety of ways. They may block gamma-secretase completely, or they may alter the activity of the enzyme so that less A-beta 42 and more of the alternative, soluble, forms of A-beta., such as A-beta 37, 38 or 39 are produced. Such modulators may thereby retard or reverse the development of AD.

Compounds are known, such as indomethacin, ibuprofen and sulindac sulphide, which reduce the production of A-beta 42 while increasing the production of A-beta 38 and leaving the production of A-beta 40 constant.

In some embodiments, compounds of the present invention are useful gamma-secretase modulators. In some embodiments, compounds of the present invention modulate the action of gamma-secretase such that amyloid-beta (1-42) peptide production in a patient is attenuated. In certain embodiments, compounds of the present invention modulate the action of gamma-secretase so as to selectively attentuate amyloid-beta (1-42) peptide production in a patient. In some embodiments, such selective attenuation occurs without significantly lowering production of the total pool of Abeta, or the specific shorter chain isoform amyloid-beta (1-40) peptide. In some embodiments, such selective attenuation results in secretion of amyloid beta which has less tendency to self-aggregate and form insoluble deposits, is more easily cleared from the brain, and/or is less neurotoxic. In some embodiments, the ability of compounds of the present invention to modulate gamma-secretase is beneficial in that there is a reduced risk of side effects with treatment resulting from, e.g., minimal disruption of other gamma-secretase controlled signaling pathways.

In some embodiments, compounds of the present invention are gamma-secretase modulators useful for treating a patient suffering from AD, cerebral amyloid angiopathy, HCHWA-D, multi-infarct dementia, dementia pugilistica or traumatic brain injury and/or Down syndrome.

In some embodiments, one or more compounds of the present invention are administered to a patient suffering from mild cognitive impairment or age-related cognitive decline or pre-symptomatic AD or prodromal or predementia AD (Dubois et al The Lancet Neurology 10 (2010) 70223-4 A favourable outcome of such treatment is prevention or delay of the onset of AD. Age related cognitive decline and mild cognitive impairment (MCI) are conditions in which a memory deficit is present, but other diagnostic criteria for dementia are absent (Santacruz and Swagerty, American Family Physician, 63 (2001), 703-13). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE.

In some embodiments, compounds of the present invention are useful for modulating and/or inhibiting amyloid-beta (1-42) peptide production in a patient. Accordingly, compounds of the present invention are useful for treating, or lessening the severity of, disorders associated with amyloid-beta (1-42) peptide production in a patient.

In some embodiments, the compounds of the present invention are useful for modulating and/or inhibiting amyloid-beta (1-40) peptide production in a patient. Accordingly, the compounds of the present invention are useful for treating, or lessening the severity of, disorders associated with amyloid-beta (1-40) peptide production in a patient. In some embodiments, compounds of the present invention do not modulate and/or inhibit amyloid-beta (1-40) peptide production in a patient.

In some embodiments, the compounds of the present invention are useful for modulating and/or inhibiting amyloid-beta (1-38) peptide production in a patient. Accordingly, the compounds of the present invention are useful for treating, or lessening the severity of, disorders associated with amyloid-beta (1-38) peptide production in a patient.

In some embodiments, the compounds of the present invention are useful for reducing both amyloid-beta (1-42) and amyloid beta (1-38). In some embodiments, the compounds of the present invention are useful for reducing amyloid-beta (1-42) and raising amyloid beta (1-38).

The compounds, extracts, and compositions, according to the method of the present invention, may be administered using any amount and any route of administration effective for treating or lessening the severity of a neurodegenerative disorder. The exact amount required will vary from subject to subject, depending on the species, age, and general condition of the subject, the severity of the infection, the particular agent, its mode of administration, and the like.

In certain embodiments, the present invention provides a method for modulating and/or inhibiting amyloid-beta (1-42) peptide production in a patient, wherein said method comprises administering to said patient a provided compound, or a pharmaceutically acceptable composition comprising said compound. In other embodiments, the present invention provides a method of selectively modulating and/or inhibiting amyloid-beta (1-42) peptide production in a patient, wherein said method comprises administering to said patient a provided compound, or a pharmaceutically acceptable composition thereof. In still other embodiments, the present invention provides a method of reducing amyloid-beta (1-42) peptide levels in a patient, wherein said method comprises administering to said patient a provided compound, or a pharmaceutically acceptable composition thereof. In other embodiments, the present invention provides a method for reducing amyloid-beta (1-42) peptide levels in a cell, comprising contacting said cell with a provided compound. Another embodiment provides a method for reducing amyloid-beta (1-42) in a cell without substantially reducing amyloid-beta (1-40) peptide levels in the cell, comprising contacting said cell with a provided compound. Yet another embodiment provides a method for reducing amyloid-beta (1-42) in a cell and increasing one or more of amyloid-beta (1-37) and amyloid-beta (1-39) in the cell, comprising contacting said cell with a provided compound.

As used herein, the term “reducing” or “reduce” refers to the relative decrease in the amount of an amyloid-beta achieved by administering a provided compound as compared to the amount of that amyloid-beta in the absence of administering a provided compound. By way of example, a reduction of amyloid-beta (1-42) means that the amount of amyloid-beta (1-42) in the presence of a provided compound is lower than the amount of amyloid-beta (1-42) in the absence of a provided compound.

In still other embodiments, the present invention provides a method for selectively reducing amyloid-beta (1-42) peptide levels in a patient, wherein said method comprises administering to said patient a provided compound, or a pharmaceutically acceptable composition thereof. In certain embodiments, the present invention provides a method for reducing amyloid-beta (1-42) peptide levels in a patient without substantially reducing amyloid-beta (1-40) peptide levels, wherein said method comprises administering to said patient a provided compound, or a pharmaceutically acceptable composition thereof.

In certain embodiments, the present invention provides a method for reducing amyloid-beta (1-42) peptide levels in a patient and increasing one or more of amyloid-beta (1-37) and amyloid-beta (1-39), wherein said method comprises administering to said patient a provided compound, or a pharmaceutically acceptable composition thereof.

In certain embodiments, the present invention provides a method for reducing amyloid-beta (1-42) peptide levels in a patient and increasing amyloid-beta (1-38), wherein said method comprises administering to said patient a provided compound, or a pharmaceutically acceptable composition thereof. In certain embodiments, the present invention provides a method for reducing amyloid-beta (1-42) peptide levels in a patient and decreasing amyloid-beta (1-38), wherein said method comprises administering to said patient a provided compound, or a pharmaceutically acceptable composition thereof.

The term “increasing” or “increase,” as used herein in reference to an amount of an amyloid-beta, refers to the relative rise in the amount of an amyloid-beta achieved by administering a provided compound (or contacting a cell with a provided compound) as compared to the amount of that amyloid-beta in the absence of administering a provided compound (or contacting a cell with a provided compound). By way of example, an increase of amyloid-beta (1-37) means that the amount of amyloid-beta (1-37) in the presence of a provided compound is higher than the amount of amyloid-beta (1-37) in the absence of a provided compound. For instance, the relative amounts of either of amyloid-beta (1-37) and amyloid-beta (1-39) can be increased either by an increased production of either of amyloid-beta (1-37) and amyloid-beta (1-39) or by a decreased production of longer amyloid-beta peptides, e.g., amyloid-beta (1-40) and/or amyloid-beta (1-42). In addition, it will be appreciated that the term “increasing” or “increase,” as used herein in reference to an amount of an amyloid-beta, refers to the absolute rise in the amount of an amyloid-beta achieved by administering a provided compound. Thus, in certain embodiments, the present invention provides a method for increasing the absolute level of one or more of amyloid-beta (1-37) and amyloid-beta (1-39), wherein said method comprises administering to said patient a provided compound, or a pharmaceutically acceptable composition thereof. In other embodiments, the present invention provides a method for increasing the level of one or more of amyloid-beta (1-37) and amyloid-beta (1-39), wherein the increase is relative to the amount of longer amyloid-beta peptides, e.g., amyloid-beta (1-40) and/or amyloid-beta (1-42), or total amyloid-beta, wherein said method comprises administering to said patient a provided compound, or a pharmaceutically acceptable composition thereof.

One of ordinary skill in the art will appreciate that overall ratio of amyloid-beta peptides is significant where selective reduction of amyloid-beta (1-42) is especially advantageous. In certain embodiments, the present compounds reduce the overall ratio of amyloid-beta (1-42) peptide to amyloid-beta (1-40) peptide. Accordingly, another aspect of the present invention provides a method for reducing the ratio of amyloid-beta (1-42) peptide to amyloid-beta (1-40) peptide in a patient, comprising administering to said patient a provided compound, or a pharmaceutically acceptable composition thereof. In certain embodiments, the ratio of amyloid-beta (1-42) peptide to amyloid-beta (1-40) peptide is reduced from a range of about 0.1 to about 0.4 to a range of about 0.05 to about 0.08.

In other embodiments, the present invention provides a method for reducing the ratio of amyloid-beta (1-42) peptide to amyloid-beta (1-40) peptide in a cell, comprising contacting the cell with a provided compound. In certain embodiments, the ratio of amyloid-beta (1-42) peptide to amyloid-beta (1-40) peptide is reduced from a range of about 0.1 to about 0.4 to a range of about 0.05 to about 0.08.

According to one aspect, the present invention provides a method for treating or lessening the severity of a disorder associated with amyloid-beta (1-42) peptide, wherein said method comprises administering to said patient a provided compound, or a pharmaceutically acceptable composition thereof. Such disorders include neurodegenerative disorders such as Alzheimer's disease, Parkinson's disease, and Down's syndrome.

Such disorders also include inclusion body myositis (deposition of A-beta in peripheral muscle, resulting in peripheral neuropathy), cerebral amyloid angiopathy (amyloid in the blood vessels in the brain), and mild cognitive impairment and pre-symptomatic, prodromal or predementia AD.

“High A-beta42” is a measurable condition that precedes symptomatic disease, especially in familial patients, based on plasma, CSF measurements, and/or genetic screening or brain imaging. This concept is analogous to the relationship between elevated cholesterol and heart disease. Thus, another aspect of the present invention provides a method for preventing a disorder associated with elevated amyloid-beta (1-42) peptide, wherein said method comprises administering to said patient a provided compound or a pharmaceutically acceptable composition thereof.

In other embodiments, the present invention provides a method for treating diseases where A-beta amyloidosis may be an underlying aspect or a co-existing and exacerbating factor, wherein said method comprises administering to said patient a provided compound, or a pharmaceutically acceptable composition thereof.

In still other embodiments, the present invention provides a method for treating a disorder in a patient, wherein said method comprises administering to said patient a provided compound, or a pharmaceutically acceptable composition thereof, and wherein said disorder is Lewy body dementia (associated with deposition of alpha-synuclein into Lewy bodies in cognitive neurons; a-synuclein is more commonly associated with deposits in motor neurons and the etiology of Parkinson's disease), Parkinson's disease, cataract (where a-beta is aggregating in the eye lens), age-related macular degeneration, Tauopathies (e.g. frontotemporal dementia), Huntington's disease, ALS/Lou Gerhig's disease, Type 2 diabetes (IAPP aggregates in pancreatic islets, is similar in size and sequence to A-beta and having type 2 diabetes increases risk of dementia), transthyretin amyloid disease (TTR, an example of this disease is in heart muscle contributing to cardiomyopathy), prion disease (including Creutzfeldt-Jakob disease, Gerstmann-Sträussler-Scheinker syndrome, fatal familial insomnia, and kuru), and CJD.

In some embodiments, the present invention provides a method for treating a disorder in a patient, wherein said method comprises administering to said patient a provided compound, or a pharmaceutically acceptable composition thereof, and wherein said disorder is mild cognitive impairment, pre-symptomatic AD, prodromal or predementia AD, Trisomy 21 (Down Syndrome), cerebral amyloid angiopathy, degenerative dementia, Hereditary Cerebral Hemorrhage with Amyloidosis of the Dutch-Type (HCHWA-D), Creutzfeld-Jakob disease, prion disorders, amyotrophic lateral sclerosis, progressive supranuclear palsy, head trauma, stroke, Down syndrome, pancreatitis, inclusion body myositis, other peripheral amyloidoses, diabetes and atherosclerosis, cerebral amyloid angiopathy, HCHWA-D, multi-infarct dementia, and/or dementia pugilistica, or traumatic brain injury.

In other embodiments, the present invention provides a method for treating or lessening the severity of Alzheimer's disease in a patient, wherein said method comprises administering to said patient a provided compound, or a pharmaceutically acceptable composition thereof.

Without wishing to be bound by any particular theory, it is believed that the present compounds are modulators of gamma-secretase which selectively reduce levels of amyloid-beta (1-42). Accordingly, another embodiment of the present invention provides a method of modulating gamma-secretase in a patient, comprising administering to said patient a provided compound, or pharmaceutically acceptable composition thereof. In certain embodiments, the present compounds are inhibitors of gamma-secretase. Said method is useful for treating or lessening the severity of any disorder associated with gamma-secretase. Such disorders include, without limitation, neurodegenerative disorders, e.g. Alzheimer's disease. In some embodiments, such disorders include cerebral amyloid angiopathy, HCHWA-D, multi-infarct dementia, dementia pugilistica, traumatic brain injury and/or Down syndrome.

The Notch/Delta signaling pathway is highly conserved across species and is widely used during both vertebrate and invertebrate development to regulate cell fate in the developing embryo. See Gaiano and Fishell, “The Role of Notch in Promoting Glial and Neural Stem Cell Fates” Annu. Rev. Neurosci. 2002, 25:471-90. Notch interacts with the gamma-secretase complex and has interactions with a variety of other proteins and signaling pathways. Notch1 competes with the amyloid precursor protein for gamma-secretase and activation of the Notch signaling pathway down-regulates PS-1 gene expression. See Lleo et al, “Notch1 Competes with the Amyloid Precursor Protein for γ-Secretase and Down-regulates Presenilin-1 Gene Expression” Journal of Biological Chemistry 2003, 48:47370-47375. Notch receptors are processed by gamma-secretase acting in synergy with T cell receptor signaling and thereby sustain peripheral T cell activation. Notch1 can directly regulate Tbx21 through complexes formed on the Tbx21 promoter. See Minter et al., “Inhibitors of γ-secretase block in vivo and in vitro T helper type 1 polarization by preventing Notch upregulation of Tbx21,” Nature Immunology 2005, 7:680-688. In vitro, gamma-secretase inhibitors extinguished expression of Notch, interferon-gamma and Tbx21 in TH1-polarized CD4+ cells. In vivo, administration of gamma-secretase inhibitors substantially impeded TH1-mediated disease progression in the mouse experimental autoimmune encephalomyelitis model of multiple sclerosis suggesting the possibility of using such compounds to treat TH1-mediated autoimmunity See Id. Inhibition of gamma-secretase can alter lymphopoiesis and intestinal cell differentiation (Wong et al., “Chronic Treatment with the γ-Secretase Inhibitor LY-411,575 Inhibits β-Amyloid Peptide Production and Alters Lymphopoiesis and Intestinal Cell Differentiation” Journal of Biological Chemistry 2004, 26:12876-12882), including the induction of goblet cell metaplasia. See Milano et al., “Modulation of Notch Processing by g-Secretase Inhibitors Causes Intestinal Goblet Cell Metaplasia and Induction of Genes Known to Specify Gut Secretory Lineage Differentiation” Toxicological Sciences 2004,82:341-358.

Strategies that can alter amyloid precursor protein (“APP”) processing and reduce the production of pathogenic forms of amyloid-beta without affecting the release of Notch intracellular domain (NICD) following the processing of Notch are highly desirable. See Wanngren, J., et al., Second generation gamma secretase modulators exhibit different modulation of Notch beta and amyloid beta production, J. Biol. Chem. 2012, article in press; Okochi, M., et al., Secretion of the Notch-1 amyloid beta-like peptide during Notch signaling, J. Biol. Chem. 2006, 281, 7890-7898. Moreover, as described above, the inhibition of gamma-secretase has been shown in vitro and in vivo to inhibit the polarization of Th cells and is therefore useful for treating disorders associated with Th1 cells. Th1 cells are involved in the pathogenesis of a variety of organ-specific autoimmune disorders, Crohn's disease, Helicobacter pylori-induced peptic ulcer, acute kidney allograft rejection, and unexplained recurrent abortions, to name a few.

According to one embodiment, the invention relates to a method of inhibiting the formation of Th1 cells in a patient comprising the step of administering to said patient a compound of the present invention, or a composition comprising said compound. In certain embodiments, the present invention provides a method for treating one or more autoimmune disorders, including irritable bowel disorder, Crohn's disease, rheumatoid arthritis, psoriasis, Helicobacter pylori-induced peptic ulcer, acute kidney allograft rejection, multiple sclerosis, or systemic lupus erythematosus, wherein said method comprises administering to said patient a provided compound, prepared according to methods of the present invention, or a pharmaceutically acceptable composition comprising said compound.

In certain embodiments, the present invention provides a method for modulating and/or inhibiting amyloid-beta peptide production, without affecting the release of Notch intracellular domain (NICD) following the processing of Notch are highly desirable, in a patient, wherein said method comprises administering to said patient a provided compound, or a pharmaceutically acceptable composition comprising said compound.

In certain embodiments, the present invention provides a method for inhibiting amyloid-beta (1-42) peptide production, without affecting the release of Notch intracellular domain (NICD) following the processing of Notch are highly desirable, in a patient, wherein said method comprises administering to said patient a provided compound, or a pharmaceutically acceptable composition comprising said compound.

In certain embodiments, the present invention provides a method for reducing amyloid-beta (1-42) peptide levels in a patient and increasing one or more of amyloid-beta (1-37) and amyloid-beta (1-39), without affecting the release of Notch intracellular domain (NICD) following the processing of Notch are highly desirable, wherein said method comprises administering to said patient a provided compound, or a pharmaceutically acceptable composition thereof.

Accordingly, another aspect of the present invention provides a method for reducing the ratio of amyloid-beta (1-42) peptide to amyloid-beta (1-40) peptide in a patient, without affecting the release of Notch intracellular domain (NICD) following the processing of Notch, comprising administering to said patient a provided compound, or a pharmaceutically acceptable composition thereof. In certain embodiments, the ratio of amyloid-beta (1-42) peptide to amyloid-beta (1-40) peptide is reduced from a range of about 0.1 to about 0.4 to a range of about 0.05 to about 0.08.

The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient,” as used herein, means an animal, preferably a mammal, and most preferably a human.

Various functions and advantages of these and other embodiments of the present invention will be more fully understood from the examples described below. The following examples are intended to illustrate the benefits of the present invention, but do not exemplify the full scope of the invention.

### EXEMPLIFICATION

The following experimentals describe preparation of exemplary compounds of the present invention. Melting points are uncorrected. ¹H and ¹³C NMR spectra were measured at 400 and 100 MHz respectively in CDCl₃ or pyridine-d5. Chemical shifts are downfield from trimethylsilane (TMS) as internal standards, and J values are in hertz. Mass spectra were obtained on API-2000, or Hewlett Parkard series 1100 MSD with ESI technique. All solvents used were reagent grade. Gamma-oryzanol was purchased from ChemPacific Corporation (Baltimore, Md., USA). The black cohosh extract was obtained as a custom order from Hauser Pharmaceuticals. This extract is substantially equivalent to the USP preparation of black cohosh extract, in which about 50% aqueous ethanol is used to extract powdered root and then concentrated to near dryness. Other abbreviations include: Ac₂O (acetic anhydride), DMAP (dimethylaminopyridine), PhI(OAc)₂ (iodosobenzene diacetate), PDC (pyridinium dichromate), TFAA (trifluoroacetic acid), DMDO (dimethyldioxirane), DIPEA (N,N-Diisopropylethylamine), RB (round-bottom), TLC (thin layer chromatography), MeOH (methanol), MeOD (methanol d-4), /—PrOH (isopropanol), TBDMS (tert-butyldimethylsilyl-), TBS (tert-butyldimethylsilyl-), DHEA (dehydroepiandrosterone), TBHP (tert-butylhydroperoxide), DMSO (dimethylsulfoxide), KOt-Bu (potassium tert-butoxide), MS (mass spectrometry), Mom-Cl (Chloromethyl methyl ether), EtOAc (ethyl acetate), M.P. (melting point), EtPPh₃I (ethyltriphenylphosphonium iodide), Et₃N (triethyl amine), mCPBA (met[alpha]-chloroperbenzoic acid), BF₃OEt₂ (trifluoroborane etherate), EtOH (ethanol), HPLC (high performance liquid chromatography), LCMS (liquid chromatography mass spectrometry), NMR (nuclear magnetic resonance).

General procedures: Reagents were acquired commercially and used without further purification except where noted. LC/MS spectra were acquired using an Agilent MSD with electrospray ionization and Agilent 1100 series LC with a Zorbax C-18 column (2.1×30 mm, 3.5 micron particle size). Standard LC conditions utilized CH₃CN with 0.1% formic acid as the organic phase and water containing 0.1% formic acid as the aqueous phase, and were run as follows: Flow rate 1.000 mL/min; 0-1.80 minutes 2-98% organic-aqueous; 1.80-3.75 minutes 98% organic-aqueous, 3.75-3.76 minutes 98-2% organic-aqueous; 3.76-4.25 minutes 2% organic-aqueous. LC/MS samples included here are of reaction mixtures pre-workup unless otherwise noted. Automatic integration over the entire non-background signal is included here, and selected key masses for individual regions have been added manually. NMR spectra were acquired using a Varian 400 MHz instrument and are acquired in CDCl₃.

### Example 1

### Step E1-1:

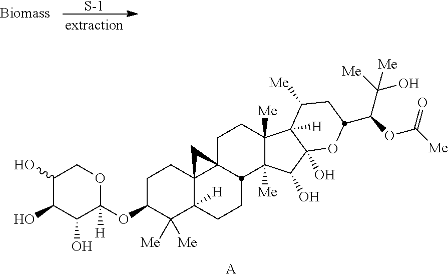

The black cohosh biomass was first dried and ground to a suitable particle size usually ranging from about 0.1 to about 1.0 mm³. This may be accomplished by passage through a chipper or a grinding mill. The ground biomass (1.88 kg) was extracted with tech grade methanol (9.4 L) at 50° C. for 2 hours. Other polar solvent such as an alcohol, preferably 95% ethanol could also be used. The extraction could also be done at RT for 22 hours. The extract solution was filtered through Celite using a basket centrifuge. The filter cake was rinsed with tech grade methanol and the filtrates were combined. The clear, homogeneous, dilute methanol extract was concentrated under vacuum with a maximum temperature of 33° C. reached, which provided 1.3 L of concentrated solution with suspended solids visible. The concentrated extract was added slowly to 5% KCl solution in water (5.2 L) and the resulting mixture was cooled to 4° C. and held for 2 hours. Other salts can also be used, including but not limited to, (NH₄)₂SO₄, K₂SO₄, NaCl, etc. The concentration of salt in water can range from 3% to 30%. The holding time can range from 2 to 24 hours. The precipitate containing compound A was formed, which was collected using a centrifuge and rinsed with water. An aqueous salt solution can also be used to rinse the solid, including but not limited to, 0-30% (NH₄)₂SO₄, K₂SO₄, KCl, NaCl, etc. Sometimes Celite was added as filter aid to facilitate the filtration. The collected solids were transferred to a dryer which provided 71 g of dry solid. The type of dryer which can be used includes but is not limited to a spray dryer, drum dryer, etc.

The above solid was taken up in 210 mL of CH₂Cl₂ and the obtained slurry was stirred at RT for 1 h, followed by addition of 268 mL of 10% NaCl. The organic phase was collected and the aqueous layer was extracted again with 70 mL of CH₂Cl₂. The combined organic phase was evaporated to dryness, which afforded 56.7 g of solid, which contains 13% of A by HPLC-ELSD analysis.

HPLC analysis conditions:
Column. Phenomenex Luna C18(2), 3 μm, 4.6 mm×150 mm;
Flow rate: 1.0 mL/min;
Detector: ELSD, Temp.: 55° C., Gain 11;
Gradient:

| | Water | Acetonitrile | Methanol |
|---|---|---|---|
| Time | (v/v %) | (v/v %) | (v/v %) |
| 0.0 | 40 | 35 | 25 |
| 10.0 | 25 | 50 | 25 |
| 15.0 | 5 | 70 | 25 |
| 18.0 | 5 | 70 | 25 |
| 18.1 | 40 | 35 | 25 |
| 23.0 | 40 | 35 | 25 |

Rt of A1 (xyloside)=7.9 min
Rt of A2 (arabinoside)=7.2 min

### Step E1-2:

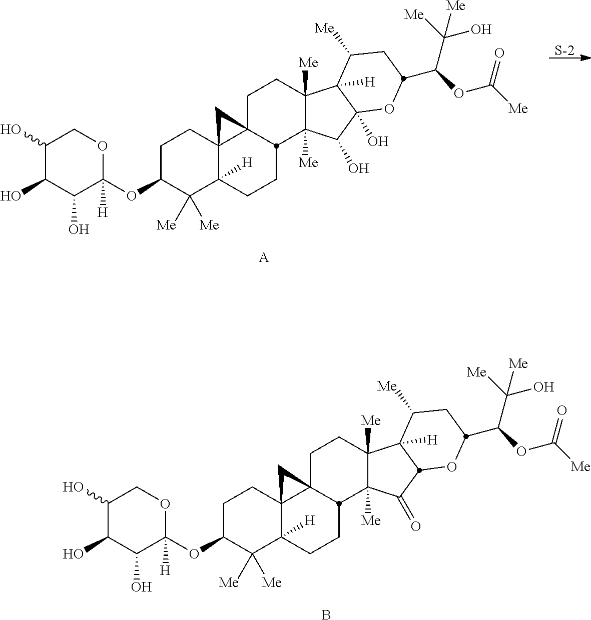

Method S2-A: To a solution of the solid obtained from S-1 (20.3 g, 13% A) in CH₂Cl₂ (162 mL) was added ZrCl₄ (1.32 g) at 20° C. in three portions over 1 h. The mixture was stirred at 20° C. for additional 35 min and Celite (7.1 g) was added, followed by addition of Et₃N (5 mL) within 5-15 min. The solids were filtered off and washed with CH₂Cl₂ (100 mL). The filtrates were combined and washed with half saturated NaHCO₃ (100 mL). The aqueous layer was back extracted with CH₂Cl₂ (25 mL). All the organic layers were combined and evaporated to dryness, which afforded crude product B (19.16 g). Purification of the crude on SiO₂ (100 g) with 0-7% MeOH/CH₂Cl₂ provided B (4.07 g) in 58% purity based on HPLC-ELSD analysis. Precipitation of the solid in EtOH/water (41 mL/49 mL) at 5° C. provided an upgraded compound B (2.4 g) in 83.3% purity by HPLC-ELSD analysis. HPLC-ELSD conditions: see above in S-1. Rt of B1 (xyloside)=7.2 min. Rt of B2 (arabinoside)=6.7 min.

Method S2-B: Alternatively, the solid obtained from S-1 (32 g, 13% A) was dissolved in DMSO (70 mL), filtered through Celite and purified by reverse phase chromatography with C-18 column (40-63 μm, 18.2 cm×45 cm) using 60-70% MeOH/water as eluents. The fractions were analyzed using the analytical HPLC conditions described above. The selected fractions were combined and concentrated to about half of the original volume (1.1 L). NaCl (143 g) was added and the resulting mixture was extracted with CH₂Cl₂ (2×340 mL). The combined organic phase was concentrated to dryness. Further drying in vacuo provided 4.0 g of solid A in 62.3% purity by HPLC-ELSD analysis. To a solution of the above solid (62.3% A, 4.0 g) in CH₂Cl₂ (80 mL) was added ZrCl₄ (200 mg) at 20° C. The mixture was stirred at 20° C. for 75 min and Celite (4.0 g) was added followed by addition of Et₃N (0.83 mL) within 5-15 min. The solids were filtered off and washed with CH₂Cl₂ (51 mL). The filtrates were combined and most solvent was removed by distillation at 30-40° C. The residue was azeotroped with EtOH to remove the rest of CH₂Cl₂. Precipitation of the residue in EtOH/H₂O (9/11) provided compound B (1.2 g) in 96% purity by HPLC-ELSD analysis. HPLC-ELSD conditions: see above in S-1. Rt of B1 (xyloside)=7.2 min. Rt of B2 (arabinoside)=6.7 min.

### Step E1-3:

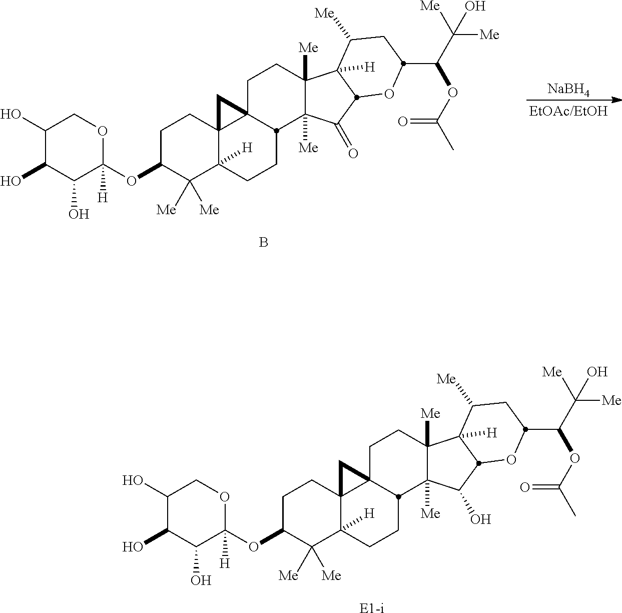

Sodium borohydride (1.72 g, 45.3 mmol) was added to ethanol (20 mL) in a 500 mL round-bottom flask and allowed to stir for 10 min. EtOAc (100 mL) was added followed by B (10 g, 15.1 mmol) at room temperature. After 1 h LC/MS showed good conversion and some acetate cleavage. HCl was added carefully over several minutes with cooling in an ice bath (evolution of hydrogen!), the solution stirred for 10 min, and the solution was partitioned between 200 mL each CH₂Cl₂ and water. The layers were separated, then the aqueous layer extracted with 100 mL CH₂Cl₂ (×2) and the combined organic layers were dried over Na₂SO₄, filtered, and the solvent removed under reduced pressure to give the product (8.2 g, 82%) which was purified by chromatograph column.

### Step E1-4:

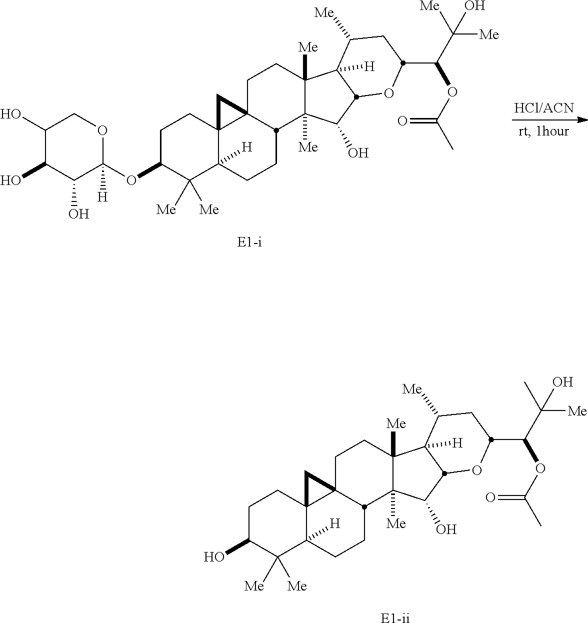

HCl (conc) (15 mL) was added to E1-i (8.2 g, 12.3 mmol) in CH₃CN (60 mL) and allowed to stir for 1 h. The reaction mixture was partitioned between 400 mL each CH₂CL₂ and water. The organic layer was washed with NaHCO₃, then dried over Na₂SO₄, filtered, concentrated and purified by biotage chromatography to give pure E1-ii (2.8 g, 43%).

### Step E1-5:

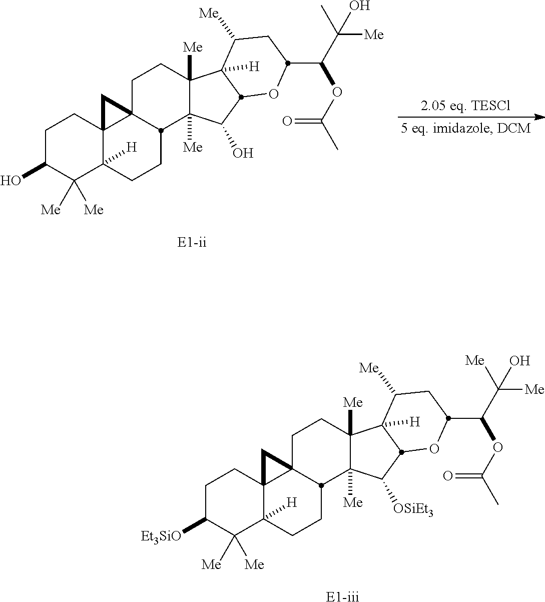

TESCl (486 mg, 3.24 mmol) was added to triol (840 mg, 1.58 mmol) plus imidazole (537 mg, 7.89 mmol) in CH₂Cl₂ (10 mL). TLC showed good balance of conversion to the desired bis-silyl ether and tri-protection after 1 hour. Water (50 mL) was added and the mixture was extracted with CH₂Cl₂ (3×20 mL). The combined organic layers were dried over Na₂SO₄, filtered and concentrated to give E1-iii which was purified by chromatography (750 mg, 62.5%).

### Step E1-6:

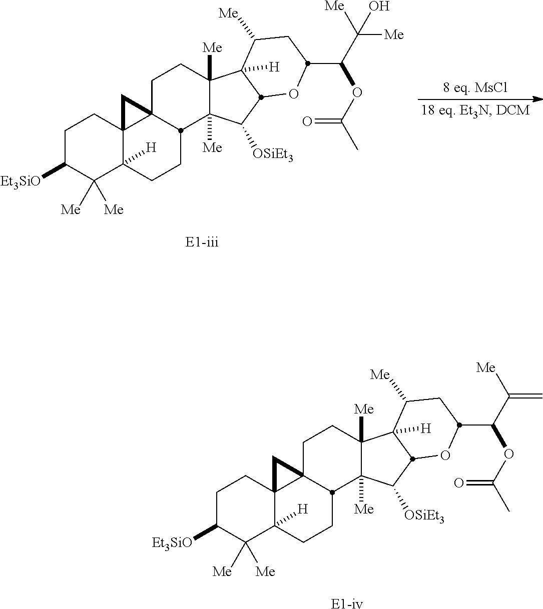

To a solution of E1-iii (2.3 g, 3.03 mmol) and Et₃N (5.51 g, 54.54 mmol) in dry DCM (10 mL) chilled in an ice water bath was added MsCl (2.71 g, 24.21 mmol) dropwise slowly. Then the mixture was stirred at room temperature for 1 hour. TLC showed the reaction was completed. Water (50 mL) was added and the mixture was extracted with CH₂Cl₂ (20 mL×3). The combined organic layers were washed with brine, dried over Na₂SO₄, filtered and concentrated to give E1-iv which was purified by column chromatography (1.37 g, 61%).

### Step E1-7:

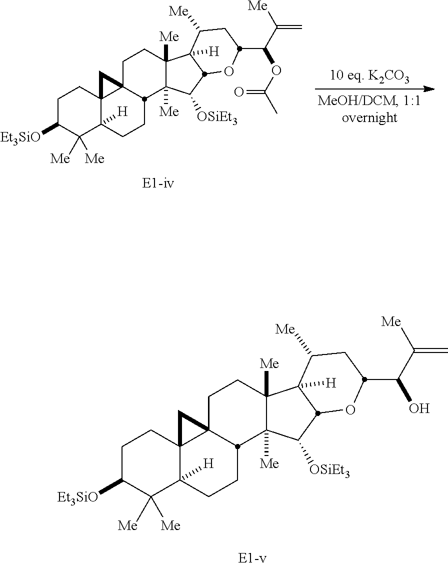

To a solution of E1-iv (1.37 g, 1.85 mmol) in CH₂Cl₂ (15 mL) and MeOH (15 mL) was added K₂CO₃ (2.49 g, 18.5 mmol). Then the mixture was stirred at room temperature for 12 hours. TLC showed the reaction was completed. The mixture was concentrated and dissolved in CH₂Cl₂ (150 mL). The organic layer was washed with water (20 mL×3), brine, dried over Na₂SO₄, filtered and concentrated to give E1-v, which was used for the next step directly (1.28 g, 99%).

### Step E1-8:

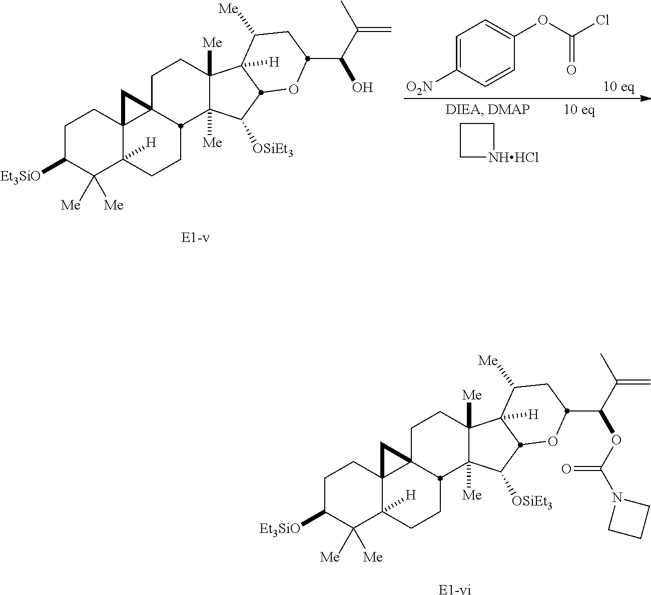

4-Nitrophenyl chloroformate (860 mg, 4.28 mmol) was added to DIEA (277 mg, 2.14 mmol), DMAP (523 mg, 4.28 mmol) and E1-v (300 mg, 0.43 mmol) in dry CH₂Cl₂ (2 mL) under N₂ and allowed to stir for 12 hours. Azetidine hydrochloride salt (200 mg, 2.15 mmol) was added. Then the mixture was stirred at room temperature for another 2 hours. TLC showed the reaction was completed. Water (30 mL) was added and the mixture was extracted with CH₂Cl₂ (15 mL×3). The organic layers were combined, washed with brine, dried over Na₂SO₄, filtered and concentrated to give E1-vi which was purified by column chromatography (180 mg, 54%).

### Step E1-9:

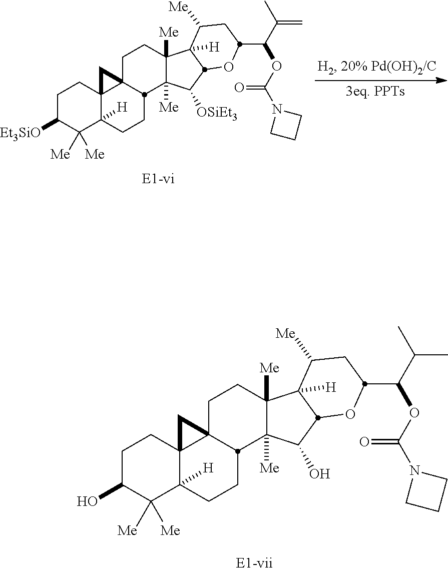

A solution of E1-vi (180 mg, 0.23 mmol) in MeOH (10 mL) and EtOAc (2 mL) was treated with 20% Pd(OH)₂ on carbon (wet) (36 mg). The reaction mixture was stirred under H₂ (1 atm) at room temperature for 30 min. TLC showed the reaction was finished. Then the mixture was filtered and solvent was removed in vacuo to give the residue. To the residue in CH₂Cl₂ (2 mL) and MeOH (2 mL) was added PPT_{S} (173 mg, 0.69 mmol). Then the mixture was stirred at room temperature for 30 minutes. TLC showed the reaction was completed. Solvent was removed in vacuo and the obtained residue was purified by column chromatography to give E1-vii (120 mg, 94%).

### Step E1-10:

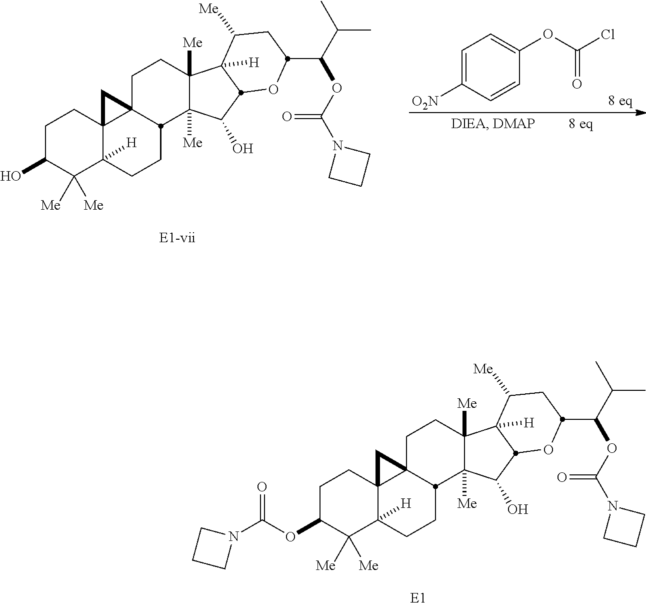

4-Nitrophenyl chloroformate (8 equiv.) was added to DIEA (5 equiv.), DMAP (8 equiv.) and E1-vii (1 equiv.) in dry CH₂Cl₂ (1 mL) under N₂ and stirred for 12 hours. TLC showed that the starting material disappeared. At this stage, the salt of an amine, usually a hydrochloride salt (3 equiv.), was added. The mixture was stirred at room temperature for another 2 hours. When TLC showed that the reaction was completed, water was added and the mixture was extracted with CH₂Cl₂. The organic layers were combined, washed with brine, dried over Na₂SO₄, filtered and concentrated to give a compound of formula I which was purified by preparative HPLC.

TABLE 2
Exemplary compounds

| Compound | Corresponding | (m/z) [M + H]⁺ | Exact |
|---|---|---|---|
| # | Amine in Step E1-10 | or [M + Na]⁺ | MS |
| I-1 | 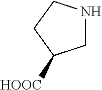 | 699.9 | 698.45 |
| I-2 | 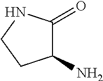 | 684.3 | 683.45 |
| I-3 | 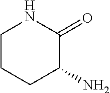 | 698.3 | 697.47 |
| I-4 | 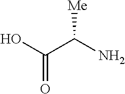 | 673.3 | 672.43 |
| I-5 | 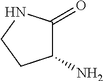 | 684.3 | 683.45 |
| I-6 | 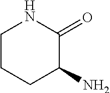 | 720.7 | 697.47 |
| I-7 | 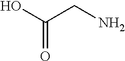 | 673.3 | 672.43 |
| I-8 | 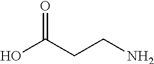 | 673.4 | 672.43 |
| I-9 | 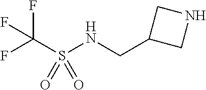 [SMILES: O=S(=O)(NCC1CNC1)C(F)(F)F] | 802.3 | 801.42 |
| I-10 | 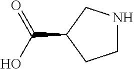 | 699.3 | 698.45 |
| I-11 | 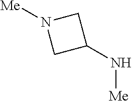 | 684.4 | 683.49 |
| I-12 | 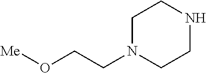 | 728.4 | 727.51 |
| I-13 | 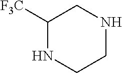 | 738.4 | 737.46 |
| I-14 | 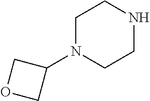 | 726.5 | 725.5 |
| I-15 | 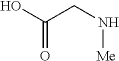 | 673.3 | 672.43 |

### Biological Assays: Aβ-42, Aβ-40, and Aβ-38

Assays were conducted to determine the ability of a Compound of Formula I to modulate Aβ-40, Aβ-40, and Aβ-38.

### Procedure:

μELISA Plates:

Human (6E10) Ab 3-PLEX ELISA kits were purchased from Meso Scale Discovery Labs, 9328 Gaither Road, Gaithersburg, Md. 20877 (Catalog Number K15148E-3). Plates with capture antibodies were blocked for 1-2 hours at room temperature with 150 μL of the manufactures blocking reagent.

Conditioned Media:

Cultured 2B7 cells in 96 well plate with 250 μL of media per well until confluent;

Prepared serial dilutions of compounds in DMSO at 100× the final desired concentration;

Washed wells with 2B7 cells 1× with 250 μL of media;

Diluted DMSO stocks 1:100 into media:

Added 250 μL of media containing compounds (1% DMSO) to wells with 2B7 cells for 5 hours at 37° C.

ELISA Sample Prep:

Diluted conditioned media: 1 part media with 1% DMSO and 1 part blocking buffer;

150 μL of the 250 μL of conditioned media were used.

Standard Curve Sample Prep:

Prepared per manufacturer's protocol (see above).

Seven point standard curve samples were prepared that contained Aβ-42, Aβ-40, and Aβ-38. The highest concentration of Aβ-42 and Aβ-38 was 3,000 μg/mL and the highest concentration of Aβ-40 was 10,000 pg/mL. Subsequent serial dilutions were 1:3 and the final composition of each sample was 1 part blocking buffer and 1 part cell medium containing 1% DMSO.

Overnight Sample Incubation:

Blocked plates are washed 5× with MSD wash buffer with a plate washer;

25 μL of detection antibody and blocker G reagent in MSD blocking solution was added;

25 μL of samples (1 part conditioned media containing 1% DMSO and 1 part MSD blocking buffer) were then added;

Plates were incubated overnight at 4 degrees C. or 2 hours at room temperature.

Final Wash and Readout:

Washed wells 5× with MSD wash buffer;

Added 150 μL 2×MSD read buffer;

Read with MSD imager.

Buffers:

All reagents were in kit.

Data Analysis:

Aβ peptide levels for each peptide were calculated from the standard curve using the MSD software provided with the MSD 2400 Imager. Percent vehicle values for each compound dosage were then calculated and fit to a 4 parameter curve generating IC50 values.

Cell Viability:

To the remaining 100 μL of conditioned media in the tissue culture plate was added 100 μL of CellTiter-Glo reagent from Promega. The plate was placed on an orbital rotator operating at 500 rpms for 2 minutes. The plate was left static for 10 minutes and then 150 μL of the lysates were transferred to a white plate and read in a luminometer.

Biological Activity Data (Table 3):

Compounds having an activity designated as “A” provided an IC₅₀<100 nM; compounds having an activity designated as “B” provided an IC₅₀ of 100-500 nM; compounds having an activity designated as “C” provided an IC₅₀ of 501-1000 nM; compounds having an activity designated as “D” provided an IC₅₀ of 1001-5000 nM; and compounds having an activity designated as “E” provided an IC₅₀>5000 nM.

TABLE 3
Biological Assays: Aβ-38, Aβ-40, and Aβ-42

| | | Aβ 42 | | Aβ 38 | | Aβ 40 |
|---|---|---|---|---|---|---|
| Compound | Aβ 42 | DR IC50 | Aβ 38 | DR IC50 | Aβ 40 | DR IC50 |
| # | IC50 | Inflection | IC50 | Inflection | IC50 | Inflection |
| I-1 | D | D | D | N/A | E | E |
| I-2 | B | N/A | C | N/A | E | N/A |
| I-3 | B | B | C | D | E | E |
| I-4 | C | N/A | D | C | E | N/A |
| I-5 | B | N/A | B | B | E | E |
| I-6 | B | N/A | B | B | E | E |
| I-7 | B | N/A | D | D | E | N/A |
| I-8 | C | C | C | C | E | N/A |
| I-9 | D | N/A | D | N/A | N/A | N/A |
| I-10 | D | N/A | D | N/A | E | N/A |
| I-11 | A | A | B | B | E | E |
| I-12 | C | N/A | D | N/A | E | N/A |
| I-13 | D | N/A | D | N/A | N/A | N/A |
| I-14 | C | N/A | C | N/A | N/A | N/A |
| I-15 | B | N/A | C | N/A | E | N/A |

1. A compound of formula I: 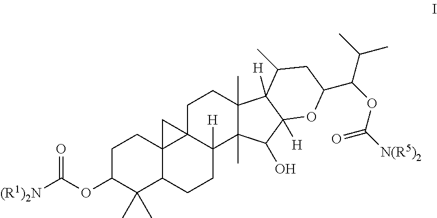
or a pharmaceutically acceptable salt thereof, wherein:
each R¹ is independently hydrogen, straight or branched C₁₋₆ alkyl, 3-6 membered cycloalkyl, or 3-6 membered saturated heterocyclyl having 1-2 heteroatoms independently selected from oxygen, nitrogen, or sulfur, wherein each R¹ is optionally substituted with 1-4 R² groups, or:
two R¹ groups are taken together to form a 3-7 membered heterocyclic ring having 0-2 heteroatoms independently selected from oxygen, nitrogen, or sulfur in addition to the nitrogen atom where R¹ groups are attached, wherein the ring is optionally substituted with 1-3 R² groups; or
two R¹ groups are taken together to form a 6-10 membered fused or spiro heterocyclic ring having 0-4 heteroatoms independently selected from oxygen, nitrogen, or sulfur in addition to the nitrogen atom where R¹ groups are attached, wherein the ring is optionally substituted with 1-3 R² groups;
each R² is independently, halogen, —C(O)N(R)₂, —N(R)₂, —OR, —NR³S(O)₂R³, oxo, —C(O)OR, C₁₋₃ alkyl optionally substituted with 1-3 R³ groups, 3-6 membered cycloalkyl, 3-6 membered saturated heterocyclyl having 1-2 heteroatoms independently selected from oxygen, nitrogen, or sulfur, wherein the cycloalkyl and heterocyclyl are each optionally substituted with 1-3 R³, or:
two R² groups on the same carbon atom are taken together to form an optionally substituted 3-6 membered saturated carbocyclic or a 3-7 membered heterocyclic ring having 1-2 heteroatoms independently selected from oxygen, nitrogen, or sulfur;
each R³ is independently hydrogen, halogen, —OR, 3-6 membered cycloalkyl, —N(R)₂, C₁₋₃ alkyl optionally substituted with 1-3 R⁴ groups, or:
two R³ groups on the same carbon atom are taken together to form an optionally substituted 3-6 membered saturated carbocyclic or a 3-7 membered heterocyclic ring having 1-2 heteroatoms independently selected from oxygen, nitrogen, or sulfur;
each R⁴ is independently hydrogen, halogen, C₁₋₃ alkyl optionally substituted with 1-3 —OR groups, 3-6 membered cycloalkyl, or:
two R⁴ groups on the same carbon atom are taken together to form an optionally substituted 3-6 membered saturated carbocyclic or a 3-7 membered heterocyclic ring having 1-2 heteroatoms independently selected from oxygen, nitrogen, or sulfur;
each R⁵ is independently hydrogen or C₁₋₃ alkyl, or:
two R⁵ groups on the same nitrogen atom are taken together to form an optionally substituted 3-5 membered heterocyclic ring; and
each R is independently hydrogen, C₁₋₄ aliphatic, or:
two R groups on the same nitrogen atom are taken together to form an optionally substituted 4-8 membered saturated or partially unsaturated ring.

2. The compound according to claim 1, wherein said compound is of formula I-a 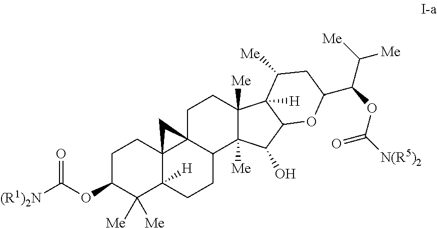
or a pharmaceutically acceptable salt thereof.

3. The compound according to claim 1, wherein one of R¹ is hydrogen.

4. The compound according to claim 1, wherein each R¹ is independently a straight or branched C₁₋₆ alkyl wherein the C₁₋₆ alkyl is optionally substituted with 1-4 R² groups.

5. The compound according to claim 4, wherein each R¹ is independently methyl, ethyl, n-propyl, isopropyl, 2,2-dimethylpropyl, 2-methylpropyl, tert-butyl, wherein each R¹ group is optionally substituted with 1-2 R² groups.

6. The compound according to claim 1, wherein each R¹ is independently a 3-6 membered cycloalkyl.

7. The compound according to claim 6, wherein each R¹ is independently cyclohexyl, cyclopentyl, cyclobutyl, or cyclopropyl.

8. The compound according to claim 1, wherein each R¹ is independently a 3-6 membered saturated heterocyclyl having 1-2 heteroatoms independently selected from oxygen, nitrogen, or sulfur.

9. The compound according to claim 8, wherein each R¹ is independently piperidinyl, pyrrolidinyl, azetidinyl, aziridinyl, tetrahydro-2H-pyranyl, tetrahydrofuranyl, or oxetanyl.

10. The compound according to claim 1, wherein two R¹ groups are taken together to form a 3-7 membered heterocyclic ring having 0-2 heteroatoms independently selected from oxygen, nitrogen, or sulfur in addition to the nitrogen atom where R¹ groups are attached, wherein the ring is optionally substituted with 1-3 R² groups.

11. The compound according to claim 10, wherein the 3-7 membered heterocyclic ring is selected from:

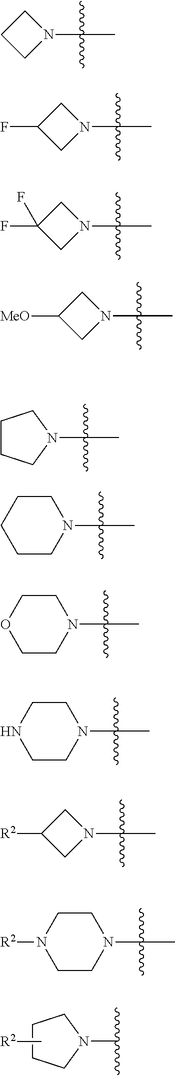

12. The compound according to claim 1, wherein one of R⁵ is hydrogen.

13. The compound according to claim 1, wherein each R⁵ is independently C₁₋₃ alkyl.

14. The compound according to claim 1, wherein two R⁵ groups on the same nitrogen atom are taken together to form an optionally substituted 3-5 membered carbocyclic ring.

15. The compound according to claim 14, wherein —N(R⁵)₂ is selected from:

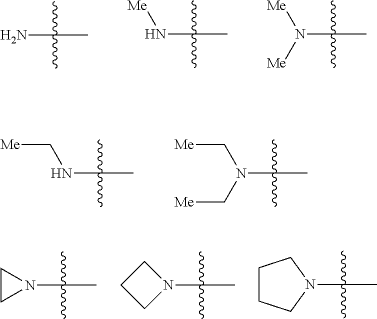

16. The compound according to claim 1, of formula II: 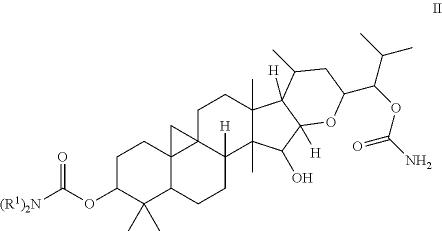
or a pharmaceutically acceptable salt thereof.

17. The compound according to claim 16, wherein one of R¹ is hydrogen.

18. The compound according to claim 16, wherein each R¹ is independently methyl, ethyl, n-propyl, isopropyl, 2,2-dimethylpropyl, 2-methylpropyl, tert-butyl, wherein each R¹ group is optionally substituted with 1-2 R² groups.

19. The compound according to claim 16, wherein each R¹ is independently cyclohexyl, cyclopentyl, cyclobutyl, or cyclopropyl.

20. The compound according to claim 16, wherein each R¹ is independently 3-6 membered saturated heterocyclyl having 1-2 heteroatoms independently selected from oxygen, nitrogen, or sulfur.

21-51. (canceled)
